(19) United States
(12) Patent Application Publication
(10) Pub. No.: US 2014/0004146 A1
(43) Pub. Date: Jan. 2, 2014

(54) METHOD FOR PRODUCING VIRUS-LIKE PARTICLE BY USING DROSOPHILA CELL AND APPLICATIONS THEREOF

(71) Applicants: Paul Zhou, Shanghai (CN); Yufeng Song, Shanghai (CN); Fan Zhou, Shanghai (CN); Lifei Yang, Shanghai (CN); Cheguo Cai, Shanghai (CN); Heng Ding, Shanghai (CN)

(72) Inventors: Paul Zhou, Shanghai (CN); Yufeng Song, Shanghai (CN); Fan Zhou, Shanghai (CN); Lifei Yang, Shanghai (CN); Cheguo Cai, Shanghai (CN); Heng Ding, Shanghai (CN)

(73) Assignee: INSTITUT PASTEUR OF SHANGHAI, CHINESE ACADEMY OF SCIENCES, Shanghai (CN)

(21) Appl. No.: 14/005,767

(22) PCT Filed: Mar. 19, 2012
(86) PCT No.: PCT/CN2012/000334

§ 371 Date: Sep. 17, 2013

(30) Foreign Application Priority Data
Mar. 17, 2011 (CN) 201110065251.4

(51) Int. Cl. C07K 14/005

(52) U.S. Cl. CPC C07K 14/005
USPC 424/204.1; 435/69.3; 435/348

(57) ABSTRACT

The present invention relates to methods and applications of using Drosophila cells to produce virus-like particles. Virus-like particles of enveloped viruses produced by the methods of the present invention have proteins correctly expressed, cleaved, and assembled. Ultimately, virus-like particles having good immunogenicity are obtained. The present invention also provides recombinant cells expressing virus-like particles and compositions containing virus-like particles.

### CROSS-REFERENCE TO RELATED APPLICATIONS

This is a national stage application based on PCT/CN2012/000334, filed on Mar. 19, 2012, which claims priority to Chinese Patent Application No. 201110065251.4, filed on Mar. 17, 2011. This application claims the priority of these prior applications and incorporates their disclosures by reference in their entireties.

### BACKGROUND OF INVENTION

1. Field of the Invention

The present invention belongs to the field of biotechnology. More specifically, the present invention relates to methods and applications of using Drosophila cells to produce virus-like particles.

2. Background Art

Virus-like particle (VLP) containing intact and biologically active envelope protein antigens can often induce good immune response without any adjuvant. In addition, because VLP does not possess genetic material, it does not have an ability to replicate and it is not infectious. Compared with the attenuated and inactivated vaccines, VLP production and vaccination are safer. In fact, as shown in many animal model experiments, VLP has already been a new type of vaccines with a great development potential. At present, VLP vaccines against human HPV and HBV have been on the market. It should be noted here that HPV is a non-enveloped virus and HBV VLP only contains HBV surface protein, but does not contain viral core. Thus, compared with VLP derived from enveloped viruses, VLP derived from the above are easier for mass production.

Currently, VLP of enveloped viruses, such as influenza and HIV, are produced from insect cells transfected with recombinant baculovirus vectors and from yeast cells or mammalian cells (239T cells, COS cells, and Vero cells) co-transfected with DNA plasmids encoding viral envelope proteins and core proteins. Morphology of VLP released to cell supernatant is very similar to that of the wild type influenza virus and HIV virus. However, at present, the majority of studies that use mammalian cells or yeast cells for VLP production depend on transient transfection systems. VLP, thus produced, can only satisfy small animal research, but is not sufficient for large animal or human studies. To overcome these limitations, stable mammalian cell transfectants for VLP production have been subsequently generated. Although quantity produced by these methods are higher than that by transient transfection systems, it still cannot meet the quantity required for large animal and human studies.

HIV VLP and influenza virus VLP may be produced by using insect cells transfected with recombinant baculovirus vectors. VLP, thus produced, can induce humoral and cellular immune responses. Influenza virus VLP can protect mice from influenza virus challenges. In the meantime, HA, NA and M1 have been simultaneously constructed into a single recombinant baculovirus vector. After transfected into insect cells, VLP, thus produced, can similarly induce excellent immune response and immune protection. However, there are three major drawbacks of using methods that use insect cells transfected with recombinant baculovirus vectors to produce VLP. First, cell supernatants not only contain VLP, but also recombinant baculovirus. Even though sucrose density gradient methods show the particle density of recombinant baculovirus is lower than that of influenza VLP, it is difficult to separate these two. Therefore, VLP, thus prepared, is mixed with many recombinant baculovirus. This could seriously affect the immunogenicity of VLP and interfere with VLP quality control. Secondly, influenza HA and HIV envelope proteins are initially synthesized in endoplasmic reticulum to first form HA₀ and gp160 precursors in mammalian cells. They are then cleaved by endogenous proteases to become HA1 and HA2, or gp120 and gp41. However, to date, all the related studies show that VLP produced by insect cells transfected with recombinant baculovirus vectors contain HA₀ and gp160 precursors, indicating that HA₀ and gp160 can not be properly cleaved in such transfected cells. Although it is not clear whether the presence of pre-cleaved HA₀ precursor affects the immunogenicity and immune protection of VLP, but, in HIV studies, it has been determined that the effect of gp160 precursor on the infectiousness and immunogenicity of HIV is critical. Finally, after cells infected with recombinant baculovirus, these cells can only survive and express virus-like particles for a limited period of time (usually 5-7 days). Therefore, when expression to produce virus-like particles is needed, new recombinant baculovirus is required for infecting cells, thus, resulting in differences in quantity and quality in the expression of each batch of virus-like particles.

Viruses can be divided into enveloped viruses and non-enveloped viruses based on the presence or the absence of envelope in morphology. Enveloped viruses obtain envelope in two major ways. Most enveloped viruses obtain envelop when budding on plasma membrane of host cell. They include, but not limited to, influenza virus, human immunodeficiency virus, paramyxoviruses, Borna disease virus, rabies virus, Ebola virus, etc. At present, the commonly accepted processes of obtaining envelope by enveloped viruses include the following four steps. First, viral nucleocapsid is formed in the nucleus or cytoplasm. Second, a large number of viral transmembrane glycoprotein aggregate on cell membrane. The third step, the cytoplasmic portions of these transmembrane glycoproteins interact with viral nucleocapsid, possibly through a direct interaction, or an indirect interaction, or interact through intermediate cytoskeletal proteins. Finally, the plasma membrane carrying transmembrane viral glycoprotein gradually engulfs viral nucleocapsid particles. When virus nucleocapsid particles are completely engulfed by lipid bilayers, virus particles are formed and depart from the infected cell by budding. Another type of viruses obtain their envelops from cytoplasmic membrane, such as membranes of endoplasmic reticulum or Golgi complex. They include, but not limited to, flaviviruses, hepadnaviruses, rubella virus, coronavirus, Rift Valley fever virus and Bunyaviridae, etc. The main processes are, first, viral nucleocapsid is formed in the nucleus or cytoplasm. Second, a large number of viral transmembrane glycoprotein aggregate on cytoplasmic membrane. The third step, cytoplasmic membrane carrying trans-cytoplasmic membrane glycoproteins gradually engulfs viral nucleocapsid. When virus nucleocapsid particles are completely engulfed, enveloped viruses are formed by budding within endoplasmic reticulum. Fourth, the budding enveloped viruses exit endoplasmic reticulum, enter transport vesicles, transport vesicles carrying enveloped viruses enter Golgi complex, and enveloped viruses pass through Golgi complex. Fifth, Golgi complex engulfs viruses inside exocytic vesicles. Sixth, enveloped viruses are released outside the cell through exocytosis. Also, herpesvirus may obtain envelope from inner nuclear membrane. Envelope fuses with outer nuclear membrane and released into cytoplasm. Subsequent processes are similar to that of the above. The mechanism of obtaining envelope is unclear for some viruses, such as iridovirus and poxvirus. But, studies found that their envelopes may not be derived from any pre-existing membrane. In the 1970s, scientists first discovered the sera of infected patients contain not only viral particles, but also virus-like particles similar to viral particles. Studies show that these are viruses resulted from defective replication assembly process. These virus-like particles fail to enclose genetic materials critical for viral integration into host genome. Up to now, elucidation of the mechanism for VLP self-assembly is completely based on the understanding of the self-replication assembly maturation process post-viral infection. Conversely, VLP model provides an excellent tool for scientists to understand the natural viral replication process. Thus, scientists' understanding of VLP production methods for these three types of viruses is also based on their natural replication maturation processes.

In summary, systems or methods of further optimizing VLP production are still needed in the field to obtain VLP with strong immunogenicity, high yield, and stable production, suitable for industrial production and clinical application.

### SUMMARY OF INVENTION

The object of the present invention is to provide methods and applications of using Drosophila cells to produce virus-like particles.

The first aspect the present invention provides a method of producing a virus-like particle of an enveloped virus, the method comprising: transforming a Drosophila cell with a nucleic acid encoding an antigenic protein of the enveloped virus, obtaining a recombinant virus-like particle producing cell; culturing the recombinant virus-like particle producing cell, from which expressing and obtaining the virus-like particle. In another preferred embodiment, the Drosophila cell is a Drosophila melanogaster S2 cell.

In a preferred embodiment, the nucleic acid comprises a nucleic acid encoding a viral core protein and a nucleic acid encoding the antigenic protein of the enveloped virus.

In another preferred embodiment, the method comprising:

(A) providing a first expression construct, comprising a nucleic acid sequence encoding the viral core protein;
(B) providing a second expression construct, comprising a nucleic acid sequence encoding the antigenic protein of the enveloped virus;
(C) transforming Drosophila S2 cells with the constructs of (A) and (B), obtaining a recombinant virus-like particle producing cell; and
(D) culturing the recombinant virus-like particle producing cell of (C), from which expressing and obtaining the virus-like particle.

In another preferred embodiment, the viral core protein is selected from: Gag protein of human immunodeficiency virus, M1 protein of influenza virus, Gag protein of simian immunodeficiency virus, Gag viral core protein of murine leukemia virus, M viral core protein of vesicular stomatitis virus, core protein VP40 of Ebola virus, M and E proteins of coronavirus, N protein of Bunia virus, core protein C of hepatitis C virus, core protein of hepatitis B virus, core protein of SARS coronavirus, and a combination thereof.

In another preferred embodiment, the enveloped virus is a virus obtaining envelope when budding on a cell membrane of a host cell.

In another preferred embodiment, the virus comprising influenza virus, human immunodeficiency virus, paramyxovirus, Borna disease virus, rabies virus, and Ebola virus.

In another preferred embodiment, the first expression construct and the second expression construct are located on an expression vector; or the first expression construct and the second expression construct are located on different expression vectors.

In another preferred embodiment, the expression vector is a non-viral vector.

In another preferred embodiment, the promoter used in the expression vector is a promoter of Drosophila cell, selected from: MT promoter or Ac5 promoter.

In another preferred embodiment, the non-viral vector is selected from: pMT/V5-His, pMT/BiP/V5-His, pMT-DEST48, or pMT/V5-His-TOPO.

In another preferred embodiment, the method further comprises: transforming Drosophila S2 cells with a nucleic acid encoding a regulator of expression of virion protein.

In another preferred embodiment, the method further comprises: transforming Drosophila S2 cells with a resistance selection gene.

In another preferred embodiment, the virus-like particle is a virus-like particle derived from an influenza virus, said method comprising:

(A1) providing a first expression construct, comprising a nucleic acid sequence encoding Gag protein of human immunodeficiency virus or a nucleic acid sequence encoding M1 protein of influenza virus;
(B1) providing a second expression construct, comprising a nucleic acid sequence encoding neuraminidase antigen of influenza virus and/or a nucleic acid sequence encoding hemagglutinin antigen of influenza virus;
(C1) transforming the Drosophila S2 cell with the constructs of (A1) and (B1), obtaining a recombinant virus-like particle producing cell; and
(D1) culturing the recombinant virus-like particle producing cell of (C1), from which expressing and obtaining the virus-like particle;
Additional conditions are: the first expression construct and the second expression construct are located on an expression vector; or the first expression construct and the second expression construct are located on different expression vectors.

In another preferred embodiment, the nucleic acid sequence encoding neuraminidase antigen of influenza virus and the nucleic acid sequence encoding hemagglutinin antigen of influenza virus of the second expression construct of the step (B1) are located on the same expression vector or located on different expression vectors.

In another preferred embodiment, the virus-like particle is a virus-like particle derived from human immunodeficiency virus, the method comprising:

(A2) providing a first expression construct, comprising a nucleic acid sequence encoding Gag protein of human immunodeficiency virus;
(B2) providing a second expression construct, comprising a nucleic acid sequence encoding envelope protein precursor Gp160 of human immunodeficiency virus;
(C2) transforming Drosophila S2 cells with the constructs of (A2) and (B2), obtaining a recombinant virus-like particle producing cell; and
(D2) culturing the recombinant virus-like particle producing cell of (C2), from which expressing and obtaining the virus-like particle;
Additional conditions are: the first expression construct and the second expression construct are located on an expression vector; or the first expression construct and the second expression construct are located on different expression vectors.

In another preferred embodiment, it further comprises providing a third expression construct comprising a nucleic acid sequence encoding rev of human immunodeficiency virus; and transforming Drosophila S2 cells with the third expression construct and the first and the second expression constructs.

Another aspect of the present invention provides a virus-like particle obtained by any one of the methods mentioned above.

Another aspect of the present invention provides use of the virus-like particle in the manufacture of a medicament for the prevention, control, or treatment of the following diseases, disorders, or conditions: influenza, HIV, measles, respiratory syncytial virus infection, mumps, pneumonia virus infection, Borna disease, rabies, and Ebola haemorrhagic fever.

Another aspect of the present invention provides an immunogenic composition, the composition comprising:

(a) the virus-like particle; and
(b) a pharmaceutically acceptable carrier.

In another preferred embodiment, the composition further comprises: adjuvant.

Another aspect of the present invention provides a vaccine composition, the composition comprising:

(i) an antigenic protein; or an antigenic protein-expressing construct comprising an antigen nucleic acid sequence encoding the antigenic protein;
(ii) a virus-like particle, wherein the virus-like particle is obtained by any one of the methods mentioned above.

Another aspect of the present invention provides a kit used for producing a virus-like particle, comprising:

(1) an expression vector, comprising a first expression construct, comprising a nucleic acid sequence encoding a viral core protein; and a second expression construct, comprising a nucleic acid sequence encoding an antigenic protein of an enveloped virus; and
(2) a Drosophila S2 cell.

In another preferred embodiment, the kit of (1) further comprising: a third expression construct, comprising a nucleic acid sequence encoding a regulator of expression of virion protein; and/or a fourth expression construct, comprising a sequence of a resistance selection gene;

Additional conditions are: the first expression construct and/or the second expression construct and/or the third expression construct and/or the fourth expression construct are located on an expression vector or located on different expression vectors.

Another aspect of the present invention provides a Drosophila cell, the Drosophila cell comprising a vector for producing a virus-like particle of an enveloped virus. The Drosophila cell is preferably a Drosophila melanogaster S2 cell.

In another preferred embodiment, the vector for producing the virus-like particle of the enveloped virus comprising a nucleic acid encoding a viral core protein and a nucleic acid encoding an antigenic protein of an enveloped virus.

Other aspects of the present invention, due to the disclosure herein, would be obvious to a skilled person in the art.

### BRIEF DESCRIPTION OF DRAWINGS

FIG. 1. Schematic diagrams show the plasmids used for the preparation of HIV VLP (pDOL). Gp160 (HIVenv), rev (HIVrev), and Gag (HIVgag), respectively, represent a gene encoding HIV-1 Gp160 (pDOL) protein, a gene encoding Rev protein, and a gene encoding the full length Gag protein. Selection Marker indicates plasmid (a plasmid vector containing Hygromycin B resistance gene) used for selecting positive clones.

FIG. 2. Detection of HIV gp120 and gag expression in cell lysate and supernatant of S2 cells co-transfected with pMT-bip-HIVenv, pAC-HIVgag, pMT-rev, and pCoBlast with and without CdCl₂ induction. S.P.: supernatant; 44: stably transfected Drosophila S2 clone #44; I: induced; NI: non-induced; M: molecular weight markers; PC: positive control (lysate of 293T cells transfected with the same plasmids); 6D or 3D: supernatant collected 6 or 3 days after induction. gp120 is a fragment obtained from cleavage of gp160. P55gag is the full-length gag. The primary antibodies for detecting P55gag and gp120 are, respectively, a specific anti-HIV-1 gag p24 antibody (purchased from AIDS Reagents and Depositary program, NIAID, NIH; clone 183-H12) and a specific anti-HIV-1 gp120 antibody (purchased from Advanced BioScience Laboratories, Inc. Item No. 4317). The secondary antibody is AP-conjugated anti-mouse antibody (purchased from Promega, Item No. s3721).

FIG. 3. Evaluation of HIV VLP (pDOL) samples by Western blot after sucrose gradient. P.C.: positive control (lysate of 293T cells transfected with the same plasmids); marker: molecular weight markers; unfraction: concentrated samples without separation by sucrose gradient centrifugation; the number (such as, 1.24, etc.) of each lane, respectively, denotes the total amount of sample in each lane before TCA precipitation. The primary antibodies for detecting P55gag and gp120 are, respectively, a specific anti-HIV-1 gag p24 antibody (purchased from AIDS Reagents and Depositary program, NIAID, NIH; clone 183-H12) and a specific anti-HIV-1 gp120 antibody (purchased from Advanced BioScience Laboratories, Inc. Item No. 4317). The secondary antibody is AP-conjugated anti-mouse antibody (purchased from Promega, Item No. s3721).

FIG. 4. HIV VLP (pDOL) observed under electron microscopy.

FIG. 5. Detection of HIV-1gp120 antibody activity (serum samples with 1:1,000 dilutions) in mouse serum after immunization of HIV VLP (pDOL). The PBS control group, the DNA plasmid immunized-VLP (pDOL)/CpG/ISA720 immunization group; in which CpG and ISA720 are two immuno-adjuvant.

FIG. 6. Inhibition rate of the mouse immune serum samples at different dilutions (titers) in neutralizing the activity of homologous (pDOL) and heterologous (Q168) of pseudo-HIV-1 virus antibody. Sample number #21-24.

FIG. 7. Cytokines (IFN-γ and TNF-α) are labeled to determine the response of CD4 and CD8 T cells to HIV-1 peptides.

FIG. 8. The top figures are schematic diagrams of plasmids used for preparing influenza virus VLP. Selection Marker is a vector having blasticidin resistant gene. Plasmid construction and expression detection for influenza HA, NA, M1 and gag protein. Left side, figures A-C show HA/NA VLP having M1 as core protein. Right side, figures A-C show HA/NA VLP having HIV-1 Gag as core protein. W/O indicates without CdCl₂. The primary antibodies are, respectively, anti-HA (California 0609) pAb (purchased from eENZYME), anti-NA antibody is mouse IgG monoclonal anti-FLAG tag antibody (purchased from Sigma), anti-gag (HIV) (purchased from AIDS Reagents and Depositary program, NIAID, NIH; clone 183-H12) and anti-M1 (SZ) patient serum (purchased from and kindly provided by professor Toyota at the IPS). The secondary antibody is AP-conjugated anti-mouse antibody (purchased from Promega, Item No. s3721).

FIG. 9. Evaluation of influenza VLP samples after sucrose gradient by SDS/PAGE and Western Blot. Top panel shows the results of evaluation using anti-HIV-1 gag antibody. Middle panel shows the results of evaluation using anti-HA serum. Bottom panel shows the results of evaluation using anti-flag tag. UNF: concentrated samples without separation by sucrose gradient centrifugation. 1 to 11: samples separated by sucrose gradient centrifugation. Anti-NA antibody is anti-FLAG tag mouse IgG monoclonal antibody (purchased from Sigma), HA immune serum (CMV/R HA (Th) plasmid were injected BALB/c mice three times intramuscularly. Injection interval is 2 to 3 weeks. Two weeks after the third injection, mouse serum is collected and separated to obtain HA immune serum).

FIG. 10. Influenza VLP observed under electron microscopy.

FIG. 11. Body weight change (left panel) and survival rate (right panel) in mice challenged by homologous virus 10 MLD50.

FIG. 12. Body weight change (left panel) and survival rate (right panel) in mice challenged by heterologous virus 10 MLD50.

FIG. 13. Body weight change (left panel) and survival rate (right panel) in mice challenged by homologous virus 1,000 MLD50.

FIG. 14. Gross pathology of BALB/c mice infected with 1,000 MLD50 of homologous H5N1 virus. (a) A representative mouse lung in the PBS control group. (b) A representative mouse limb paresis in the PBS control group. (c) A representative mouse relaxed intestine and colon in the PBS control group.

FIG. 15. Lung pathology after challenged by 10 MLD50 of homologous virus. (a-d) Hematoxylin and eosin (HE) staining of lung tissues of mice four days after infection with 10 MLD50 (homologous (A/Shenzhen/406H/06, clade 2.3.4) H5N1 virus). (a) PBS control group; (b) homologous VLP-VLP group; (c) homologous DNA-DNA group; (d) heterologous DNA-VLP group.

FIG. 16. Lung pathology after challenged by 1,000 MLD50 of homologous virus. (e-h) Hematoxylin and eosin (HE) staining of lung tissues of mice four days after infection with 1,000 MLD50 (homologous (A/Shenzhen/406H/06, clade 2.3.4) H5N1 virus). (e) PBS control group; (f) homologous VLP-VLP group; (g) homologous DNA-DNA group; (h) heterologous DNA-VLP group.

FIG. 17. ELISA antibody binding reaction results of each immunization group.

FIG. 18. Sequence of HIV-1 Gag p55 (SEQ ID NO: 1).

FIG. 19. Cryo-microscopy and tomographic images of representative clone S2 (VB2) that produces HIV-1 VLP (consensus B and C). A. Cryo-cross sectional images of a representative HIV-1 VLP (consensus B and C) obtained from a linear sucrose gradient upper band. Scale bar is 100 nm. B. Cryo-cross sectional images of a representative HIV-1 VLP (consensus B and C) obtained from a linear sucrose gradient lower band. Scale bar is 100 nm. C. Cryo-microscopy and tomographic images (Z-stack) of a representative HIV-1 VLP obtained from a linear sucrose gradient lower band. Red arrow indicates the location of the spike. D. HIV-1 VLP surface model simulated based on the above cryo-microscopy and tomographic images. White dots in the figure represent positions of envelope protein spikes.

FIG. 20. ADCC and ADCVI responses of each immune serum sample produced by heterologous immunization with DNA-HIV-1 VLP (consensus B and C). A. Detection of HIV-1 envelope proteins expressed on cell surface of CEMss-CCR5 infected with HIV-1 AD8 by using pooled sera from PBS control mice and heterologous DNA-HIV-1 VLP immunized mice, and HIV-1 patient pooled sera. B. Staining CEMss-CCR5 target cells infected with HIV-1 AD8 by using PKH-26 and CFSE fluorescent dye. Co-incubating the stained target cells with spleen cells from BALB/c (E: T=50:1). Then, add serum sample with 1:50 dilution from PBS control mice and serum sample from DNA-HIV-1 VLP heterologously immunized mice. After 18 hours, measure ADCC as described in materials and methods. C. Co-incubate CEMss-CCR5 target cells infected with HIV-1 AD8 and spleen cells from BALB/c mouse (E:T=20:1). Then, add serum sample with 1:50 dilutions from PBS control mice and serum sample from DNA-HIV-1 VLP heterologously immunized mice. After 2 days, detect gag p24 protein in supernatant using ELISA, and calculate the percent inhibition according to the calculation methods described in materials and methods. D. Analysis of correlation between the percent inhibition of ADCC and ADCVI of each serum sample from PBS control mice and DNA-HIV-1 VLP heterologously immunized mice by linear regression analysis.

### DETAILED DESCRIPTION

After years of in-depth studies, the present inventors, for the first time, developed methods of producing virus-like particles using stably transfected Drosophila S2 cells. Virus-like particles of enveloped viruses produced by the methods of the present invention have proteins correctly expressed, cleaved, and assembled. Ultimately, virus-like particles having good immunogenicity are obtained.

### Terminology

As used herein, the term “animal” may be any animal that can have immune response to virus-like particles produced by virus-like particle production systems of the present invention. It includes mammal (including human, pigs, cattle, horse, sheep, mule, and other livestock, or other economic animal), poultry (such as chicken, duck, goose), bird (such as quail, pet bird), and so on. The biological virus-like particles produced by virus-like particle production systems of the present invention can be used in humans or animals. In the present invention, mice serve as test organisms, which are very close, as compared with humans, in genome composition, ontogeny, metabolism, organ anatomy, and disease pathogenesis.

As used herein, the term “antigenic protein” or “antigen” refers to proteins having immunogenicity, or proteins useful for the construction of virus-like particles. The “antigenic protein” also includes their protein variants, provided that the protein variants retain the function or activity of “antigenic protein”.

As used herein, the term “construct” refers to a nucleic acid containing a nucleic acid sequence encoding a specific protein, used for the transformation of cells. The “construct” may also include promoters or terminators operably linked to nucleic acid sequences encoding specific proteins, etc. One or more constructs may be, respectively, included in one or more expression vectors, used for the transformation of cells and expression.

As used herein, the term “promoter” or “promoter region” refers to a nucleic acid sequence, which is usually present upstream (5′end) from the coding sequence of a gene, capable of directing transcription of the nucleic acid sequence to produce mRNA. In general, the promoter or promoter region provides recognition sites for RNA polymerases and other factors required for correctly initiating transcription. In the present disclosure, the promoters or promoter regions include promoter variants obtained by insertion or deletion of their regulatory regions through random or site-directed mutagenesis, etc. Gene transcription regulated by tissue- or organ-specific promoters generally takes place only in certain specific organs or tissues.

As used herein, the term “operably linked” or “operably connected” refers to the functions of two or more nucleic acid regions or nucleic acid sequences arranged spatially. For example: a promoter region is placed at a specific location in relation to that of the nucleic acid sequence of a target gene, such that transcription of the nucleic acid sequence is directed by the promoter region, whereby the promoter region is “operably linked” to the nucleic acid sequence.

As used herein, the term “effective amount” refers to an amount that produces function or activity in humans and/or animals and is acceptable for humans and/or animals.

As used herein, the “pharmaceutically acceptable” ingredient is a substance suitable for humans and/or mammals without excessive adverse side effects (such as toxicity), i.e. having reasonable benefit/risk ratio.

As used herein, the term “pharmaceutically acceptable carrier” refers to a carrier for the administration of therapeutic agents, including various excipients and diluents. This term refers to some pharmaceutical carriers: they, by themselves, are not essential active ingredients without excessive toxicity after the application. Suitable carriers are well known to one skilled in the art. Pharmaceutically acceptable carriers in composition may contain liquid, such as water, saline, and buffer. In addition, auxiliary substances may also be present in these carriers, such as fillers, lubricants, glidants, wetting, or emulsifying agents, pH buffering substances, etc. The carriers may also contain cell transfection reagents. For vaccines, the pharmaceutically acceptable carriers may include, for example, adjuvant.

As used herein, the term “comprising,” “having,” or “including” includes “containing”, “mainly . . . composed of,” “basically . . . composed of,” and “composed of;” “mainly . . . composed of,” “basically . . . composed of,” and “composed of;” belong to the subordinate concept of “comprising,” “having,” or “including.”

### Production of Virus-Like Particles

Drosophila cells can be stably cultured in laboratory conditions (Schneider, J. Embryol. Exp. Morphol. 27:353 (1972)). Many vector systems containing specific coding sequences can be inserted into Drosophila genome through Drosophila heat shock promoters and COPIA promoters (DiNocera et al., Proc. Natl. Acad. Sci. USA 80:7095 (1983)). Moreover, mRNA from the heat shock promoters can be translated in great amount in Drosophila cells (McGarry et al., Cell 42:903 (1985)).

B. J. Bond, et al. showed the structure of actin 5C gene of Drosophila melanogaster (B. J. Bond et al, Mol. Cell. Biol., 6 (6): 2080 (1986)). The report also discussed two start sites of actin 5C gene. These two sites are fused inside the promoter sequence. Bacterial chloramphenicol acetyltransferase gene was inserted into Drosophila melanogaster host cells.

Expression of E. coli gal K gene in Drosophila cell lines is regulated by Drosophila melanogaster promoter (H. Johansen et al, 28th Annual Drosophila Conference, p. 41 (1987)).

Hygromycin B selection systems have been discussed in many literature (A. Vanderstraten et al, Proceedings of the 7th International Conference on Invertebrate and Fish Tissue Culture, Abstract, University of Tokyo Press, Japan, (1987); A. Vanderstraten et al, in “Invertebrate and Fish Tissue Culture”, Eds. Y. Kuroda et al, Japan Scientific Societies Press, Tokyo, pp. 131-134, (1988)).

The present invention is not limited to any specific Drosophila cell lines. Preferably, Drosophila cell line used in the present invention is Drosophila melanogaster cell line S2. S2 cells are stable polyploid Drosophila embryonic cells (Schneider, J. Embryol. Exp. Morph. 27: 353 (1972)). Transfection of Drosophila S2 cells with cDNA coding sequence of gp160 or its cleaved form gp120 or gp41, or other derivatives using DNA transfection techniques can produce a great amount of HIV env proteins. tPA expression can also achieve similar results. Many advantages of using Drosophila S2 cells include, but not limited to, high-density growth at room temperature. Stable selection systems have achieved up to 1,000 copies of expression units inserted into host cell genome.

Other Drosophila cell systems can also be used in the present invention, such as serum-free cell line-KC-O Drosophila melanogaster cell line (Schulz et al, Proc, Nat'l Acad. Sci. USA, 83: 9428 (1986)). However, previous studies have shown that it was more difficult to transfect KC-O cells than S2 cells. Another useful cell line was derived from Drosophila hydei cell line, which can be used for protein expression. However, the efficiency of protein expression is relatively low (Sinclair et al, Mol. Cell. Biol., 5: 3208 (1985)). Others Drosophila cell lines can be used in the present invention also include S1 cell line and S3 cell line.

Drosophila cells used in the present invention can be cultured in a variety of suitable culture medium, including M3 medium. M3 medium, pH 6.6, is composed of a series of balanced salt and essential amino acids. Medium preparation was described previously (Lindquist, DIS, 58: 163 (1982)). Other conventional media can also be used for culturing Drosophila cells.

Preferred promoter is Drosophila melanogaster promoter (Lastowski-Perry et al, J. Biol. Chem., 260:1527 (1985)). This inducible promoter can direct high level of transcription in the presence of CuSO₄. Drosophila melanogaster promoters used in expression systems can also maintain regulatory ability in the presence of high copy number. However, the effect of metal ions on the ability of mammalian metallothionein promoter to regulate in mammalian cells decreases as the copy number increases. In Drosophila expression systems, the preserved effectiveness of induction increases gene expression levels in the presence of high copy number.

Drosophila actin 5C gene promoter (B. J. Bond et al, Mol. Cell. Biol., 6: 2080 (1986)) is also a desirable promoter sequence. Actin 5C promoter is a constitutive promoter and does not require induction by additional metal ions. Therefore, it may be more suitable to use this promoter than Drosophila melanogaster promoter in mass production systems. Another advantage of this promoter is that cells can maintain better conditions for a long period of time without high concentrations of copper ions in medium.

Other Drosophila promoters also include inducible heat shock (Hsp70) promoter and COPIA LTR promoter. Expression level of SV40 early promoter system is lower than Drosophila promoter system. Promoters, such as arian Rous sarcoma virus LTR and simian virus (SV40 early promoter) generally used in cell expression vectors, have poorer function and expression in Drosophila cells.

Drosophila S2 cells described in the present invention are commercially available, for example, available for purchase from Invitrogen Co. In the prior art, Drosophila S2 cells have been routinely used for the expression and production of exogenous proteins. S2 cells can be grown at room temperature and do not require CO₂. Because Drosophila S2 cells can grow in suspension, they can achieve high density growth. Exogenous proteins can be expressed using inducible promoters (such as MT promoter) or stable expression promoter (such as Ac5 promoter). Moreover, a variety of exogenous signal peptides allow secretory proteins to be released normally from S2 cells.

Although Drosophila S2 cells have been used to produce a variety of exogenous proteins, there is yet a report that uses this system to produce VLP from enveloped and non-enveloped viruses. The present inventors have done a large numbers of research work and invented methods of producing virus-like particles (VLP) with high efficiency using Drosophila S2 cells, especially the production of virus-like particles directed to influenza viruses and virus-like particles directed to HIV viruses.

The present inventors, for the first time, produced virus-like particles of enveloped viruses in Drosophila S2 cells, which were transfected with nucleic acid sequences encoding viral core proteins and nucleic acid sequences encoding antigenic proteins of enveloped viruses. Preferably, the enveloped viruses are viruses obtaining their envelopes when budding on plasma membrane of host cells.

As a preferred embodiment of the present invention, Drosophila S2 cells also have transfected expression constructs containing nucleic acid sequences encoding a regulator of expression of virion protein (Rev), which contributes to more efficient formation of virus-like particles. Rev protein is a regulator of expression of virion protein. Rev protein is an important trans-activating factor regulating HIV gene replication. It has a negative regulatory effect on HIV regulatory proteins and a positive regulatory effect on virion proteins. Its main function is to promote conversion of HIV gene expression from the early (transcribing mRNA of regulatory proteins) to the late (transcribing mRNA of HIV structural proteins), and promoting the late transcription process. In the original HIV viruses with defective rev gene, only the early genes express. Only after Rev protein is added, transcription of the late genes then begins. Furthermore, Rev protein also plays a role in transporting mRNA of structural proteins into cytoplasm and that is achieved possibly by inhibiting RNA processing system in the nucleus or by enhancing RNA transporting system.

Nucleic acid sequences encoding fragments or variants of viral core proteins or antigenic proteins of enveloped virus may also be used. Fragments or variants (derivatives or analogs) refer to polypeptides basically retaining the same biological functions or activities as that of the viral core proteins or antigenic proteins of enveloped viruses. Fragments, derivatives, or analogs of viral core proteins or enveloped virus antigenic proteins may be (i) polypeptides substituted with one or more conservative or non-conservative amino acid residues (preferably, conserved amino acid residues), and such substituted amino acid residues may or may not be encoded by genetic code, or (ii) polypeptides having one or more amino acid residues with substituent groups, or (iii) polypeptides formed by fusing mature polypeptides with another compounds (such as compounds prolonging half-life of polypeptides, such as polyethylene glycol), or (iv) polypeptides formed by fusing additional amino acid sequence with polypeptide sequences (such as leader sequence or a secretory sequence or peptide sequences used for purifying polypeptides or fibrinogen sequences, or fusion proteins). According to the definitions of these fragments in the present disclosure, derivatives and analogs belong to the scope commonly known to one skilled in the art.

The definition of fragments of viral core protein or antigenic protein of enveloped viruses refers to polypeptides that still maintain all or part of the function of full-length viral core protein or antigenic protein of enveloped viruses. Under normal conditions, the fragments maintain at least 50% of full-length protein activity. In more preferred conditions, the fragments can maintain 60%, 70%, 80%, 90%, 95%, 99%, or 100% of full-length protein activity.

Nucleic acid sequences encoding fragments or variants of viral core proteins or antigenic proteins of enveloped viruses may be codon-optimized. This type of codon optimization can be designed based on preferences of Drosophila S2 cells. Some commercial software can be used for codon optimization design.

Constructs of the present invention may be (or may be derived from) expression vectors. Expression vectors of the present invention are not particularly limited, provided that they contain some elements required for protein expression, and these elements are operably linked. Any plasmids and vectors may be used as long as they can replicate and are stable in Drosophila S2 cells. One important characteristic of expression vectors typically contains replication origins, promoters, marker genes, and translation control elements. As a preferred embodiment of the present invention, in expression vectors, the promoter is a promoter of Drosophila cells, for example, selected from MT promoter or Ac5 (AC) promoter. In fact, other promoters of Drosophila cells can also be used to express viral core proteins or antigenic proteins of enveloped viruses.

Vectors containing the afore-mentioned suitable antigen nucleic acid sequences and suitable promoters or control sequences can be used to transform host cells, in which proteins are expressed, and virus-like particles are finally formed. The host cells are Drosophila S2 cells. Transformation of host cells with recombinant DNA can be performed by using conventional techniques well known to one skilled in the art, such as calcium phosphate transformation (transfection) method.

Systems of the present invention can be used to produce virus-like particles in large quantity and with high efficiency. Production methods use the afore-mentioned constructs to transform the virus-like particle producing cells to obtain recombinant virus-like particle producing cells; and culture the afore-mentioned recombinant virus-like particle production cells, from which express and obtain virus-like particles.

Systems of the present invention only use plasmid-transfected cells to obtain virus-like particles. Virus-like particles, thus produced, do not have contaminated recombinant virus.

As a preferred embodiment of the present invention, the construct is a combination of constructs includes, for example: a first construct, which includes the following operably linked elements: promoters and envelope protein precursor Gp160 of human immunodeficiency virus; a second construct, which includes the following operably linked elements: promoters and regulators of expression of virion proteins; and a third construct, which includes the following operably linked elements: promoters and core protein gag. After transformed Drosophila S2 cells with the combination of constructs, envelope protein precursor Gp160 can be correctly and appropriately cleaved to become gp120 and gp41 in S2 cells. Finally, virus-like particles can be very efficiently obtained and have very high immunogenicity.

As a preferred embodiment of the present invention, the construct is a combination of constructs includes, for example: a first construct, which includes the following operably linked elements: promoters and hemagglutinin antigen (HA) of influenza virus; a second construct, which includes the following operably linked elements: promoters and neuraminidase antigen (NA) of influenza virus; and a third construct, which includes the following operably linked elements: promoters and matrix protein M1 of influenza virus. After transformed Drosophila S2 cells with a combination of constructs, HA and NA envelope proteins can be effectively assembled into VLP, which can be released from cells with particle size very similar to that of the wild type virus, and has very high immunogenicity.

As a preferred embodiment of the present invention, the construct is a combination of constructs includes, for example: a first construct, which includes the following operably linked elements: promoters and hemagglutinin antigen (HA) of influenza virus; a second construct, which includes the following operably linked elements: promoters and neuraminidase antigen (NA) of influenza virus; and a third construct, which includes the following operably linked elements: promoters and gag core protein of human immunodeficiency virus. After transformed Drosophila S2 cells with the combination of constructs, HA and NA envelope proteins can be effectively assembled into VLP, which can be released from cells with particle size very similar to that of the wild type virus, and has very high immunogenicity.

Other forms of nucleic acid sequences having viral core proteins or antigenic proteins of enveloped virus and constructs with necessary gene expression elements (such as promoter) are also included in the present invention, as long as they can produce virus-like particles after Drosophila transformation.

Thus, virus-like particles produced by S2 system can overcome the drawbacks occurred in the production of VLP by insect cells transfected with recombinant baculovirus vectors.

### Virus-Like Particles and Compositions

The present invention also provides virus-like particles having immunogenicity, which are obtained essentially by systems and methods of producing virus-like particles according to the present invention.

MHC I and MHC II types of body immune systems elicit 1,000 or 10,000-fold stronger exogenous antigen presentation existed in particulate form than soluble monomer antigen presentation. That is, antigens existed in particulate form have stronger immunogenicity than soluble monomer antigens. MHC I and MHC II pathways of body immune systems elicit 1,000 or 10,000-fold stronger exogenous antigen presentation existed in particulate form than soluble monomer antigen presentation. That is, antigens existed in particulate form have stronger immunogenicity than soluble monomer antigens.

The present invention further provides use of virus-like particles having immunogenicity. Depending on differences in nucleic acid sequences encoding antigenic proteins, they can be used to treat different types of microbial infections. The microbial infections diseases, such as (but not limited to): cold, acquired immunodeficiency syndrome, pneumonia, hepatitis, bronchitis, herpes, endophthalmitis, keratitis, measles, mumps, measles, chickenpox, or shingles. When antigenic proteins are derived from influenza viruses or HIV viruses, virus-like particles are used for prevention or treatment of cold or acquired immunodeficiency syndromes.

The present invention further provides a composition having immunogenicity (preventive or therapeutic vaccines), the composition comprises: an effective amount of virus-like particles having immunogenicity according to the present invention and pharmaceutically acceptable carriers.

Pharmaceutically acceptable carrier refers to some pharmaceutical carriers: they, by themselves, are not essential active ingredients, and without undue toxicity after the application. Suitable carriers are well known to one skilled in the art. Full description related to pharmaceutically acceptable carriers can be found in Remington's Pharmaceutical Sciences (Mack Pub. Co., NJ 1991). Pharmaceutically acceptable carriers in composition may contain liquid, such as water, saline, glycerol, and sorbitol. In addition, these carriers may also have auxiliary substances, such as lubricants, glidants, wetting or emulsifying agents, pH buffering substances and stabilizers, such as albumin, etc.

Composition may be manufactured into various agent forms suitable for administration to mammals. Agent forms include, but not limited to: injection, capsule, tablet, emulsion, suppository; preferably, injection.

Animal experiments show high titer antibodies in animals after immunization with virus-like particles prepared by virus-like particles with immunogenicity of the present invention.

In use, safe and effective amounts of virus-like particles with immunogenicity of the present invention are applied to mammals (such as human), in which safe and effective amount is usually at least about 1 μg/kg body weight, and, in most cases, no more than about 10 mg/kg body weight, preferably, dosage is about 1 μg/kg body weight—about 1 mg/kg body weight. Of course, specific dosages should be considered in view of factors, such as administration route, patient health status, etc., which are within the skill of a skilled physician.

As a way of the present invention, composition also includes immuno-stimulating agents or adjuvant, for example, ISA720, CpG ODN, or ISA51, etc. However, study results of the present inventors show that, without adding any adjuvant, virus-like particles also have very good immunogenicity and heterologous DNA-VLP immunization strategies can induce better neutralizing antibody activity and CTL responses. In influenza virus challenge model, these strategies show a complete immune protection.

The following specific embodiments in combination further illustrate the present invention. It should be understood that these embodiments are merely intended to illustrate the invention and are not intended to limit the scope of the present invention. Specific conditions not indicated in experimental methods in the following embodiments are usually based on conventional conditions, such as described in Sambrook et al., Molecular Cloning: A Laboratory Manual (New York: Cold Spring Harbor Laboratory Press, 3rd edition), or conditions recommended by the manufacturers. Unless otherwise indicated, percentages and parts are calculated by weight.

Unless otherwise defined, all professional and scientific terminology used in the present disclosure has the same meaning familiar with by one skilled in the art. In addition, any methods and materials similar to or equivalent to the present disclosure can be used in the present invention. Preferred embodiments and methods described herein are for demonstration purposes only.

### I. MATERIALS AND METHODS

### Cells

Drosophila S2 cells, purchased from Invitrogen, were cultured in Express FIVE® SFM (GIBCO cat no 10486) medium added with 10% fetal calf serum (GIBCO cat no 16000) at 28° C. without CO₂, until cell concentration reached 0.5˜2×10⁶ cells per ml, which then were used for transfection.

### Plasmids Construction

HIV-1 envelope (HIVenv) and Rev gene (HIVrev) sequences, and genes encoding influenza M1, HA, and NA were generated by PCR, overlapping PCR, or recursive PCR. Recombinant plasmids were constructed as follows:

Construction of pMT-bip-HIVenv (pDOL):

Nucleic acid sequences encoding envelope proteins on the original HIV envelope protein plasmid CMV/R-Gp160 pDOL (purchased from AIDS Reagents and Depositary program, NIAID, NIH) were amplified by PCR (forward primer: ctagaattcaacagagaagctgtggg (SEQ ID NO: 2); reverse: GATGCGGCCGCTTACTTTCC (SEQ ID NO: 3)) and inserted into Bgl II and EcoR I sites of Drosophila S2 expression vector pMT-bip vector (purchased from Invitrogen).
The amino acid sequences and nucleotide sequences of HIVenv (pDOL) are, respectively, shown as Genbank Accession No. AAC82596 and AF033819.3 (5771 bp-8341 bp).

Construction of pMT-biP-160 (Consensus B and C):

HIV-1 consensus B and C env gene was used as template, and cDNA encoding HIV consensus B and C gp160 missing signal peptide (e.g., construction methods described in Kothe, D. L et al, Virology 360:218-34 and Virology 352: 438-49) was amplified by PCR, and the amplified sequences were inserted into TA vector (Invitrogen). After sequencing, the correct sequences were excised and inserted into EcoR I and Xho I sites of pMT/BiP/V5-His (Invitrogen). The plasmids, thus obtained, were named pMT/BiP-gp160 (consensus B and C), respectively.
The sequence of Gp160/consensus B is shown as SEQ ID NO: 14 or Gene Bank: DQ667594.
The sequence of Gp160/consensus C is shown as SEQ ID NO: 15 or Gene Bank: DQ401075.

Construction of pMT-bip-HIVrev:

Nucleic acid sequence encoding rev on the original HIVrev plasmid pZeoSV/rev (purchased from IPS, Institut Pasteur of Shanghai) were amplified by PCR (forward primer: ctagaattcaccatggcaggaagaag (SEQ ID NO: 4) and reverse: AGTGCGGCCGCCTATTCTTTAGC (SEQ ID NO: 5)) and then inserted into EcoR I and Not I sites of pMT-bip vector.
The amino acid sequence and nucleotide sequences HIVrev are, respectively, shown as Genbank Accession No. CAA41586 and X58781.

Construction of pMT-HIVgag and pAC-HIVgag:

Nucleic acid sequence corresponding to gag on the original HIVgag plasmid p55M (1-10) (e.g., construction methods described in Ralf Schneider et al., Journal of Virology 1997: 4892-4903) was amplified by recursive PCR methods (see Ai-Sheng Xiong et al Nature Protocol 1 (2) 2006:791) (forward primer: gtcgaattcaccatgggtgcga (SEQ ID NO: 6), reverse: GTAGCGGCCGCTTATTGTGACG (SEQ ID NO: 7)) and then inserted into EcoR I/Not I sites of pMT/V5-His A (purchased from Invitrogen) and pAc5.1/V5-His A (purchased from Invitrogen).
The nucleotide sequence HIVgag. (See FIG. 18).

Construction of pMT-bip-HA:

Nucleic acid sequence corresponding to aa19-aa568 of HA ORF on the original plasmid CMV/R HA (Th) (e.g., construction methods described in Tsai C, et al., Vaccine, 2009, Nov. 12; 27 (48):6777-90) was amplified by PCR (forward primer: GGGAGATCTTGCATCGGATACCACG (SEQ ID NO: 8), reverse: CCCGAATTCTCAG ATGCAGATTCTGC (SEQ ID NO: 9)) and then inserted into Bgl II and EcoR I sites of pMT-bip vector.
The amino acid sequence and nucleotide sequence of HA are, respectively, shown as Genbank Accession Nos. AAS65615 and AY555150.2 (1 bp-1718 bp).

Construction of pMT-bip-NA:

Nucleic acid sequence corresponding to full length NA ORF on the original NA plasmid CMV/R NA (Th)-FLAG (e.g., construction methods described in Tsai C, et al Vaccine 2009 Nov. 12; 27(48):6777-90) was amplified by PCR (forward primer: GGGGGATCCATGAATCCTAATAAGAAGATCAT (SEQ ID NO: 10), reverse: CCCGAATTCCTCACTTATCGATTGTAAAAGGCA (SEQ ID NO: 11)) and then inserted into Bgl II and EcoR I sites of pMT-bip vector.
The amino acid sequence and nucleotide sequence of HA are, respectively, shown as Genbank Accession Nos. AAS65616 and AY555151.3 (21 bp-1370 bp).

Construction of pAC-M1:

Nucleic acid sequence corresponding to full-length M1 on the original M1 plasmid CMV/R M1 (SZ) (e.g., construction methods described in Tsai C, et al Vaccine 2009 Nov. 12; 27(48):6777-90) was amplified by PCR (forward primer: CGGGAATT CACCATGAGTCTTCTAACCGAGG (SEQ ID NO: 12), reverse: CCCTCTAGATCACTTGAA TCGCTGCATCTG (SEQ ID NO: 13)) and then inserted into pGEM T easy vector (purchased from Progema). Target fragments were excised from the plasmid using EcoR I and then inserted into EcoR I sites of pAc5.1/V5-His A.
The amino acid sequence and nucleotide sequence of M1 are, respectively, shown as Genbank Accession Nos. ABO36645 and EF137707.1 (1 bp-759 bp).

### Generation of Stably Transfected Drosophila S2 Cells

To produce HIV VLP (pDOL), the present inventors constructed three plasmids: the first encodes envelope protein of HIV-1 (HIVenv (pDOL), also known as Gp160 (pDOL)). The coding gene of envelope protein was insert behind inducible MT promoter. This plasmid is pMT-bip-HIVenv (pDOL). The second (pMT-HIVgag) and the third (pAC-HIVgag) encode HIV-1 gag proteins. These coding genes were, respectively, inserted behind inducible MT promoter and stable Ac5 promoter.

Calcium phosphate methods were used to co-transfect S2 cells with plasmids (4.75 μg the first plasmid and 9.5 μg the second plasmid) or (4.75 μg the first plasmid and 9.5 μg the third plasmid) together with 4.75 μg pMT-bip-HIVrev and 1 μg pCoBlast vector plasmid containing Hygromycin B resistant gene (purchased from Invitrogen Corp., cat no R210-01 Blasticidin S HCl). 48 hours after transfection, hygromycin B was added to culture medium and incubated at room temperature without CO₂ for 2 to 3 weeks until stably transfected cell colony appeared. VLP produced from the stably transfected cell lines were named HIV VLP (pDOL). Detection of HIV-1 gag protein and envelope protein expression in cell lysates and cell culture supernatant of stably transfected cell lines with and without induction of 5 μmol CdCl₂ for 3 days was performed by Western blotting using anti-HIV-1 gag protein antibodies and anti-envelope protein monoclonal antibodies.

The present inventors also constructed pMT/BiP-gp160 (consensus B and C). S2 cells were co-transfected with 4.75 μg pMT/BiP-gp160 (consensus B and C) and 9.5 μg pAC-HIVgag, 4.75 μg pMT-bip-HIVrev, and 1 μg pCoBlast vector plasmid containing Hygromycin B resistance gene and stably transfected cell lines were obtained, as described above. VLP produced from the stably transfected cell lines was named HIV VLP (consensus B and C).

To produce influenza virus VLP, the present inventors first compared the differences between influenza virus VLP particles produced by using influenza virus M1 protein and HIV-1 gag protein as core protein. Plasmid (pAC-M1) containing encoded influenza virus M1 protein inserted behind stable Ac5 promoter and plasmid containing encoded influenza virus HA and NA proteins inserted behind inducible MT promoter (pMT-bip-HA and pMT-bip-NA) were constructed. As described previously, S2 cells were co-transfected with these plasmids, i.e., pAC-M1, pMT-bip-HA, and pMT-bip-NA (used for forming H5N1HA-NA-M1 VLP); or pAC-HIVgag, pMT-bip-HA, and pMT-bip-NA (used for forming H5N1HA-NA-HIV-1 gag VLP), respectively, and pCoBlast vector containing blasticidin resistance gene (purchased from Invitrogen) and stably transfected S2 cells were selected. Then, studies were conducted on the expression and assembly of the produced VLP.

After normal expression of transgenes in S2 cells was confirmed, limited dilution methods were used to generate stably transfected S2 cell clones. Typically, the present inventors selected 20 clones from stably transfected S2 cell lines by Western blot analysis to identify clones having high expression of virus-like particles.

### Production and Identification of Virus-Like Particles

After the establishment of stably transfected S2 cell lines capable of highly producing VLP, cells were placed in 150 cm² cell culture flasks and cultured in complete medium (10% FBS Express Five SFM medium) until cell concentration reached 500×10⁷. Then, cells were collected and placed in 2 L rotating flasks containing 500 ml fresh medium (without additional 10% FBS of Express Five SFM) and cultured. VLP was collected in cell culture supernatant and concentrated 7-fold. Then, the concentrated supernatant was centrifuged at 4° C., 20,000 rpm for 2.5 hours (Beckman Coulter, Fullerton, Calif.). Particles were re-suspended in PBS, and stored in −80° C. freezer.

Alternatively, microwave bioreactor 20/50EHT system (GE Healthcare) having WAVEPOD process control units was used and S2 clones were cultured in batch feeding culturing methods to produce HIV-1 VLP and influenza virus VLP. First, 6×10⁸ S2 cells were inoculated in 300 ml complete Express FIVE® SFM medium, placed in 2 L cell bags and cultured at 28° C. without CO₂. Initial swing speed was set at 22 rpm, swing angle at 8°. Till the third days, swing speed was increased to 26 rpm and swing angle to 9°. Cultured to the 5^{th}, 7^{th}, and 8^{th} days, 300, 200, 200 ml fresh complete medium Express FIVE® SFM were added, respectively. On the 8^{th} day, CdCl₂ to medium was added to a final concentration of 5 μM for inducing the expression of HIV-1 envelope protein and influenza virus HA, NA proteins. After 3 days of induction, culture supernatant was collected, centrifuged at 4° C. (6,000×g) for 30 minutes, and precipitate discarded. Supernatant was filtered using 0.45 μm filters. The filtered supernatant was concentrated 5-fold using QuixStand Benchtop system with 50,000 NMWC Hollow Fiber Cartridge (Model UFP-50-C-4MA). HIV-1 VLP or influenza virus VLP in the concentrated supernatant was collected by 20% sucrose buffer ultracentrifugation, resuspended in PBS, and aliquoted and stored in −80° C. freezer. Small amounts of cell supernatant were collected every 24 hours during 11-day of batch feeding culture. Cell number and activity were calculated by trypan blue exclusion test. The amount of HIV-1 VLP was determined by the detection of HIV-1 gp120 and gag p55.

To determine the properties of VLP, re-suspended particles were further centrifuged in 25-65% sucrose gradient using SW41 rotor at 25,000 rpm at 4° C. for 16 hours. 12 gradient fractions were collected from the top to the bottom of the tube and each fraction was of 0.96 ml. After TCA precipitation, separated by 12% SDS-PAGE and then transferred to PVDF membrane. Protein blot was sealed in Tris-HCl buffer containing 5% skim milk and 0.1% Tween 20, followed by incubation with primary antibody, anti-HIV-1 gag p24 antibody, anti-HIV-1 gp160 antibody, anti-HIV-1 gp120 antibody, anti-HIV-1 gp41 antibody, anti-HA antibody, anti-M1 antibody, or anti-NA antibody. Incubated with secondary antibody, which is AP-conjugated anti-mouse antibody (Promega), for color detection. The above can be performed according to manufacturer recommendation.

To further determine HIV and influenza VLP, VLP-producing cells and concentrated VLP particles were fixed with 2.5% glutaraldehyde for 30 minutes, followed by fixing with 1% osmium tetroxide. Fixed samples were dehydrated by 50-100% stepwise increase of alcohol concentrations, and then embedded in epoxy resin mixture. Polymerized at 60° C. for 72 hours. Ultrathin sections were stained with uranyl acetate and, finally, observed and photographed by transmission electron microscopy (model JEM 1230, JEOL Ltd., Japan).

To measure the amount of HIV-1 envelope protein of HIV-1 VLP, 96-well EIA/RIA plates (Costar) were coated with 1 μg/ml anti-HIV-1 gp120 C5 capture antibody (Santa cruz Cat. #4302) overnight. Coated plates were sealed with PBS containing 5% BSA at 37° C. for 1 hour. VLP-containing culture supernatant, concentrated VLP samples, or serial dilutions of standard (diluted solution: 10% BSA, 0.5% Triton X-100 in PBS) of purified gp120 protein (same preparation as described in Proc Natl Acad Sci USA 91: 8314-8) were added to the coated 96-well plates and incubated at 37° C. for 2 hours. Then, the plates were washed 5 times with PBST buffer (0.05% Tween 20 in PBS). Anti-gp120 antibody (Santa cruz Cat. #4301) at 1:2,000 dilutions was added, and then incubated for one hour. Horseradish peroxidase (HRP) linked goat anti-mouse IgG (Chemicon) at 1:5000 dilution was added. Colorimetric analysis was performed using TMB Substrate Kit (Pierce). Absorbance at 450 nm was read using spectrophotometer (BioTek Instruments, Winooski, Vt., USA). The purified gp120 protein standard curves were used to calculate the amount of HIV-1 VLP of HIV-1 envelope proteins.

To determine the amount of gag p55 of HIV-1 VLP, VLP-containing culture supernatant, concentrated VLP samples, or p24 standards as fold dilution standards (starting from 400 ng) (Aalto BioReagents) were placed on 4-12% Bis-T is gel (Invitrogen), and then transferred to PVDF membrane. PVDF membrane was sealed in 5% skim milk, followed by detection using anti-gag p24 antibody. Antigen can be directly shown by horseradish peroxidase (HRP) linked anti-mouse IgG antibody (MultiSciences) at 1:5,000 dilution and EZ-ECL substrate (Thermo). Quantity One software (Bio-Rad) was used to determine the amount of Gag of HIV-1 VLP by comparing the band density of p55 in test sample (HIV-1 VLP) and that of p24 standard sample.

### Immunization and Challenges of Highly Pathological Avian Influenza (HPAI) H5N1 Virus

After harvest, H5N1HA-NA-HIV-1 gag VLP was concentrated, dissolved in PBS overnight, and quantified by performing hemagglutination experiments (Webster, R G, Cox, N., and Stöhr, K. (2002) WHO Manual on Animal Influenza Diagnosis and Surveillance. Available from http://www.who.int/csr/resources/publications/influenza/whocdscsrncs20025). When immunization, each mouse was injected with avian influenza HA H5N1 VLP at an equivalent of 29 hemagglutination units.

Female BALB/c mice age 6-8 weeks were randomly divided into four groups, each group contained 6 mice. Table 1 shows schedule of immunization and challenge.

The first group of mice (PBS): primary and booster immunization were performed by intramuscular injection of 200 μL PBS (pH 7.4) into their two hind legs.

The second group of mice (VLP-VLP): primary and booster immunization were performed by intramuscular injection of 200 μL PBS containing avian influenza HA H5N1VLP having 29 units of hemagglutinin.

The third group of mice (DNA-DNA): primary and booster immunization were performed by intramuscular injection of 200 μL PBS containing 100 μg plasmid DNA encoding H5HA (pMT-bip-HA).

The fourth group of mice (DNA-VLP): primary immunization was performed by intramuscular injection of 200 μL PBS containing 100 μg plasmid DNA encoding H5HA, and then booster immunization was performed by intramuscular injection of 200 μL PBS containing avian influenza H5N1VLP having 29 units of hemagglutinin HA.

Primary immunization was performed on Day 7 and booster immunization was performed on Day 28. Serum samples were collected 7 days before primary immunization and 7 days after booster immunization, heat inactivated at 56° C., aliquoted and stored at −80° C.

Two weeks after booster immunization (the 42nd day), mice in each group were challenged by 50 μl 10 MLD50 (10 animals median lethal dose) homologous H5N1 virus A/Shen zhen/406H/06, subclade 2.3.4 (see N Engl J Med. 2007; 357 (14):1450-1451), and heterologous H5N1 virus A/Cambodia/P0322095/05, clade 1 (see Viruses 2009; 1(3):335-36), or 50 μl 1,000 MLD50 homologous H5N1 virus and heterologous H5N1 virus. Daily observation and recording mouse pathological features, such as drowsiness, hair loss, weight loss, etc. Four days post-challenge, one mouse selected from each group was sacrificed and lung tissues were obtained for histopathological studies. For the other mice, they were euthanized if their body weight decreased by 20% or more as compared with the original weight, and recorded as statistically dead. All operations were performed strictly in accordance with the guidelines for attending and using laboratory animals and animal welfare act issued by Ministry of Agriculture, and biosafety guidelines for microbial biochemistry laboratory issued by the Ministry of Agriculture.

TABLE 1
Schedule of immunization and challenge

| | Group | | | |
|---|---|---|---|---|
| Time | PBS | DNA-DNA | DNA-VLP | VLP-VLP |
| Day 0 | Blood drawn | Blood drawn | Blood drawn | Blood |
| | | | | drawn |
| Day 7 | PBS | DNA | DNA | VLP |
| | immunization | immunization | immunization | immu- |
| | | | | nization |
| Day 28 | PBS | DNA | VLP | VLP |
| | immunization | immunization | immunization | immu- |
| | | | | nization |
| Day 35 | Blood drawn | Blood drawn | Blood drawn | Blood |
| | | | | drawn |

| | |
|---|---|
| Day 42 | Infection with 10 MLD₅₀ H5N1 A/Shenzhen/406H/06, |
| | 10 MLD₅₀ H5N1 A/Cambodia/P0322095/05, or 1000 |
| | MLD₅₀ H5N1 A/Shenzhen/406H/06 |
| Day 46 | Sacrifice one mouse from each group for histological analysis |
| Day 42-55 | Daily measure weight and survival rate |

### Anti-HIV-1 Immunization

BALB/c mice age 6-8 week were divided into five groups and each group had 6 mice. Primary immunization of DNA was performed on Day 0 and Day 28. Booster immunization of HIV VLP (pDOL) was performed on Day 56 and Day 84, respectively.

The first group of mice (PBS): primary and booster immunization were performed by intramuscular injection of 100 μL PBS (pH 7.4) into their two hind legs.

The second group of mice (DNA-VLP): primary immunization was performed by intramuscular injection of 100 μL PBS containing 100 μg of CMV/R vector having HIV gp120 DNA and booster immunization was performed twice by subcutaneous injection of 100 μL PBS containing HIV-VLP (pDOL) having 5 μg gp120.

The third group of mice (DNA-VLP+ISA51): primary immunization was performed by intramuscular injection of 100 μg CMV/R vector containing HIV gp120 DNA and booster immunization was performed twice by subcutaneous injection of 100 μL PBS containing HIV-VLP of HIV-VLP (pDOL) having 5 μg gp120 and mixed with 5 μg CpG.

The fourth group of mice (DNA-VLP+ISA720): primary immunization was performed by intramuscular injection of 100 μg CMV/R vector containing HIV gp120 DNA and booster immunization was performed twice by subcutaneous injection of 100 pit PBS containing HIV-VLP of HIV-VLP (pDOL) having 5 μg gp120 and mixed with 5 μg ISA720 (purchased from Seppic, Paris, France).

The fifth group of mice (DNA-VLP+CpG+ISA720): primary immunization was performed by intramuscular injection of 100 μg CMV/R vector containing HIV gp120 DNA and booster immunization was performed twice by subcutaneous injection of 100 μL PBS containing HIV-VLP (pDOL) having 5 μg gp120 and mixed with 5 μg CpG and 5 μg ISA720.

Immunization program of using HIV-1 VLP (consensus B and C) is as follows: BALB/c mice age 6-8 week were divided into five groups, each group had 6 mice. Primary immunization of DNA was performed on Day 0 and Day 28 and booster immunization of HIV-1 VLP/CpG was performed on Day 56 and Day 84.

The first group of mice (PBS): primary and booster immunization were performed by intramuscular injection of 200 μL PBS (pH 7.4) into their two hind legs.

The second group of mice (DNA-VLP): primary immunization was performed with 150 μg of plasmids (containing three plasmids at 50 μg each, respectively, encoding consensus B HIV-1 gp120, consensus C HIV-1 gp120, and rev-independent HIV-1 gag) and booster immunization was performed twice by subcutaneous injection of HIV-VLP having 5 μg gp120 (consensus B and C) mixed with 5 μg CpG phosphorothioate CpG oligonucleotides (CpG-ODN 1826 5′-TCC ATG ACG TTC CTG ACG TT-3′).

Blood samples were collected from mouse orbital choroid plexus seven days before primary immunization and seven days after the second booster immunization. Specimens were agglutinated overnight. Centrifuged to collect serum samples. Aliquoted and stored at −20° C. Spleen samples were collected 10 days after the second booster immunization for intracellular cytokine staining.

Method for preparing CMV/R vector containing HIV gp120 DNA: HIV gp120 (Genebank number CAA74759.1) (see Wen, et al. Retrovirology 7:79-90, 2010; Tsai, et al Vaccine 2009 Nov. 12; 27 (48):6777-90.) was inserted into Bam HI and Sal I sites of CMV/R vector.

### Histopathological Evaluation

Lung tissues removed from infected mice were fixed by placing in 4% paraformaldehyde, and the tissues were embedded in paraffin according to conventional operation. Selected tissue sections were stained using HE method and tissue injury was examined.

### Determination of Neutralizing Activity

To determine neutralizing activity of anti-influenza virus and anti-HIV antibodies in serum before and after immunization, the present inventors seeded MDCK cells (purchased from ATCC) or TZM-bl cells (obtained from AIDS Reagents and Depositary program, NIAID, NIH) in 24-well plates at 20,000 cells per well and cultured overnight. Then, influenza HA NA pseudo-virus (prepared according to the methods described in Tsai C, et al., Vaccine 2009 Nov. 12; 27(48):6777-90), or HIV-1 pseudo-virus (homologous pDOL and heterologous Q168, prepared according to the methods described in Wen, et al., Retrovirology 7:79-90, 2010) were mixed with serum diluted two-fold and incubated at 37° C. for one hour. The mixture was added to the cells above. After overnight incubation, cells were washed with PBS and cells were cultured in complete DMEM media. Two days later, luciferase activity (influenza HA NA pseudo-virus, in the package, comes with luciferase activity) was detected in cells. Inhibition rate of neutralizing antibody activity was calculated: [value of luciferase activity (RLA) of pseudo-virus−RLA value of pseudo-virus mixed with immune serum sample at different dilutions]/RLA value of pseudo-virus. Neutralization titers (neutralizing antibody titers) refer to immune serum titer (dilution) required for inhibiting 50% or 95% of virus proliferation (IC50, IC95).

### Intracellular Cytokine Staining Methods

After spleen separated from HIV VLP immunized mice, two million cells were seeded per well in 24-well plates, while 5 ng/ml PMA and 500 ng/ml Ionomycin or 2.5 μg/ml peptide mixture (in which short peptides of env are: RGPGRAFVTI, RQAHCNISRAKWNAT, RIQRGPGRAFVTIGK, KQFINMWQEVGKAMYA; short peptides of Gag are: AMQMLKETI, EPFRDYVDRF, TTSTLQEQI, NAWVKVVEEKAFSPE, PVGEIYKRWIILGLN, VDRFYKTLRAEQASQ) and 2 μg/ml anti-mouse CD28 and 2 μg/ml CD49d antibodies (purchased from BD Biosciences, CD28 553295 and CD49d 553314) were added. After two-hour incubation at 37° C., 2 μl BD GolgiPlug™ Protein Transport inhibitor was added. Then, after four-hour incubation at 37° C., cells were transferred to FACS tubes. First, cells were seal in 1 μg mouse Fc Block (purchased from BD Biosciences 553 142) at 4° C. for 15 minutes. Then, cells were incubated with fluorescent labeled anti-CD4 and CD8 monoclonal antibody controls (CD4 BD Biosciences 553052 and CD8 BD Biosciences 553035) at 4° C. After 30 minutes, cells were treated with 200 μl BD Cytofix/Cytoperm™ solution at 4° C. After 20 minutes, cells were further stained with fluorescence-labeled anti-cytokine (TNF, IL-2 and IFNγ) antibodies or isotype control antibodies for 30-45 minutes. Then, flow cytometry was used for sample collection and data analysis.

To detect the activity of anti-HIV-1 gp120 antibody in serum, mouse sera of serial dilutions were added to kit previously coated with HIV-1 envelope protein gp120 antigen (purchased from KHB Inc. Corp.). After incubation at 37° C. for an hour, the micro-well plates were washed 5 times with washing solution, followed by adding 1:5000 dilution of HRP-labeled goat anti-mouse IgG (Chemicon International Inc., Temecula, Calif.). After incubation at 37° C. for half an hour, the micro-well plates were washed 5 times with washing solution, followed by adding 100 μl OPD peroxidase substrate (Sigma). After staining at 37° C. for ten minutes, the reaction was terminated by 50 μl 2N H₂SO₄. The values of immune response were obtained by reading excitation at 490 nm in ELISA reader.

cryo-EM and X-Ray Tomography

To purify HIV-1 VLP for EM studies, culture supernatant of stably transfected S2 clones was collected. Centrifuged at low speed (6,000×g) at 4° C. for 30 minutes. Then, filtered using 0.45 μm filter (FISHER). Placed in 20% sucrose buffer and centrifuged using SW28 rotor (25,000 rpm) for 2 hours. Pellet was resuspended in PBS. Then, placed in 25%-65% linear sucrose gradient solution. Ultracentrifuged (25,000 rpm, SW41 rotor) for 16 hours. VLP-containing portions were obtained and ultracentrifuged (25,000 rpm, SW41 rotor) for 2 hours to obtain precipitate. Pellet was resuspended in PBS. Then, placed in 30% and 45% non-linear sucrose gradient solution. Ultracentrifuged (110,000×g, MLS-50 rotor) for 3 hours. Two fuzzy bands (one band near the top of sucrose gradient, i.e., upper band, one band located at sucrose interface, i.e., lower band) were collected. Dissolved in PBS, and then filtered using 0.2 μm low protein-binding and non-heat syringe filter (cat. #PN4612, PALL). Samples were ultracentrifuged (110,000×g, MLS-50 rotor) for 2 hours. Pellet, thus obtained, was resuspended in 20 μl PBS, and stored at −80° C.

For observing VLP by cryo-EM and X-ray tomography, 3.5 μl upper and lower band samples were taken onto Quantifoil porous membrane (Quantifoil Micro Tools, GmbH, Jena, Germany), and vitrified in liquid ethane. Microscopic imaging was performed using 1,000 photographic equipment-coupled FEI Tecnai F20 electron microscope (200 kV, 38,000×, ˜2.5 μm defocus), and recorded in Gatan Ultrascan. Titan Krios (300 kV, Gatan CCD 2K×2K, 47000×) was used for Cryo-EM imaging. Pixel was set at 0.38 nm. Tilt series were collected between −62° and +60° with increments of 2° uniaxial. Defocusing was set at −8 μm. The cumulative amount was 72 e/A2.

### ADCC Test

Rapid fluorescent ADCC(RF-ADCC) test was performed according to the methods described in earlier literature (as described in Gomez-Roman, V. R. et al., J Immunol Methods 308:53-67 and Sheehy, M. E. et al., J Immunol Methods 249:99-110). Double labeling was added to 5,000 HIV-1 infected CEMss-CCR5 target cells using 5 μM PKH-26 (Sigma-Aldrich) and 0.5 μM CFSE (Molecular Probes). Labeled target cells were resuspended in RPMI 1640 medium containing 10% FBS. The 1:50 diluted preimmune and immune serum obtained from PBS immunized control mice and heterologous DNA-VLP immunized mice, natural mouse serum (negative control) or pooled HIV-1 infected patient sera (positive control) were incubated in 96-well plates at room temperature for 30 minutes. Effector cells from natural mice were added to target cells at E:T ratio of 50:1. The 96-well plates (400×g) were centrifuged for 5 min to facilitate cell-cell interactions, and then cultured at 5% CO₂, 37° C. for 4 hours. Then, cells were washed twice with PBS, and finally dissolved in 3.7% paraformaldehyde-PBS (v/v) for use in flow cytometry. Flow cytometry was BD LSRII flow cytometry. FlowJo (Tree Star Inc., USA) software was used for data analysis. ADCC death rate was determined by PKH-26^{high} number (i.e., loss of CFSE active dye and decreased cells of non-specific effect through pre-immune serum) in back-gating target cells. Every experiment contained non-label and single-labeled target cells for compensating the emission of single-labeled CFSE and PKH-26.

### ADCVI Test

Target cells and effector cells used for ADCVI test were the same as the HIV-1-infected CEMss-CCR5 cells and natural mouse cells used for ADCC test. First, viruses outside target cells (5,000) were washed away. Effector cells were added to target cells at a ratio of E:T=20:1. 1:50 dilution of pre-immune and immune serum from PBS immunized control mice and heterologous DNA-VLP immunized mice, natural mouse serum (negative control) or pooled HIV-1 infected patient sera (positive control) were added to target cells and effector cells. Control wells contained effector cells but not serum. Viral replication control wells did not contain serum and effector cells. Two days later, supernatant was collected and gag p24 protein detected by ELISA (Zeptometrix). ADCVI inhibition rate (calculated relative to the pooled pre-immune mouse serum) was calculated: percent inhibition=100 [1−(p24post)/(p24pre)], where (p24post) and (p24pre) were, respectively, p24 concentrations in supernatant in wells containing immune or preimmune serum. Each serum sample was tested three times in two independent experiments. The values obtained from two independent experiments were very close.

### II. EXAMPLE

### Example 1

### HIV VLP Production

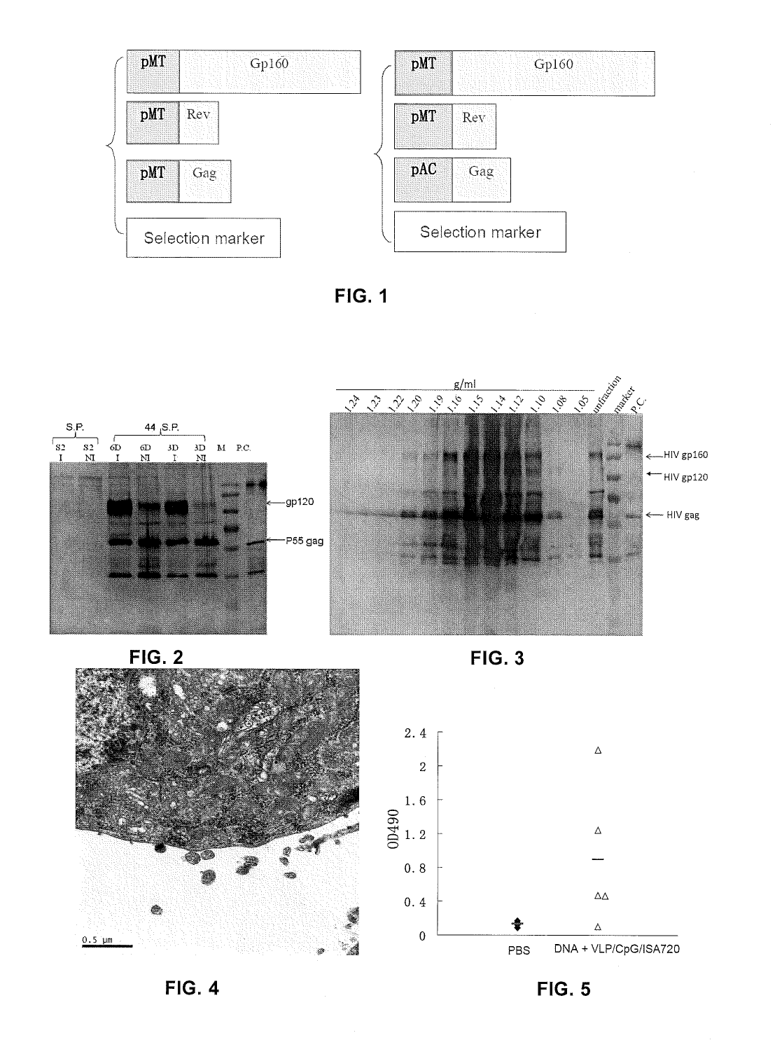

FIG. 1 shows the plasmids used for preparing HIV VLP (pDOL). Then, Drosophila S2 cells were transfected with the plasmids as described above. 48 hours after transfection, hygromycin B was added to culture medium and cultured at room temperature without CO₂ for 2 to 3 weeks until stably transfected cell colony appeared. Single-cell clones having the highest expression levels of gag protein and envelope protein were selected as VLP-producing cells.

Detect the expression of HIV-1 gag protein and envelope protein in cell lysates and cell culture supernatants of stably transfected cell lines with and without CdCl₂ induction using Western blotting. FIG. 2 shows detection of HIV gp120 and gag expression in cell lysates and supernatants of S2 cells co-transfected with pMT-bip-HIVenv, pAC-HIVgag, pMT-HIVrev and pCoBlast, with and without CdCl₂ induction. FIG. 3 shows properties of HIV-1 VLP detected by sucrose density gradient ultracentrifugation and Western blot. FIG. 4 shows a electron microscopic image of HIV-1 VLP (pDOL). The VLP particle diameter is about 100 nm. Detection results of HIV-1 VLP (consensus B and C) by Western blotting are similar to that of FIG. 2 and FIG. 3. The results show normal expression of HIV-1 gag and envelope protein (regardless of genes, pDOL or consensus B and C) in stably transfected S2 cells. Envelope proteins can also be correctly cleaved.

### Analysis of Morphology and Spike Amount of HIV-1 VLP (Consensus B and C) Produced by Drosophila S2 Cells

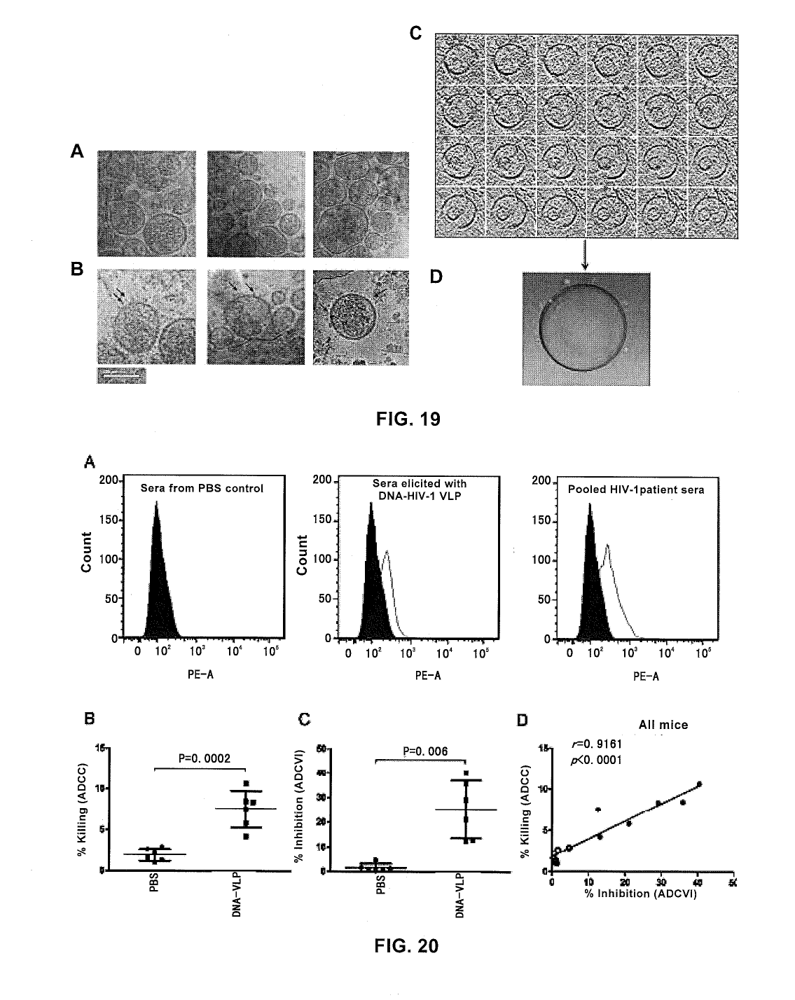

To analyze morphology of HIV-1 VLP (consensus B and C) produced by Drosophila S2 cells, first, HIV-1 VLP (consensus B and C) in culture supernatant was concentrated and purified. Use cryo-EM and X-ray tomography photography to analyze morphology and surface spikes of the purified HIV-1 VLP (consensus B and C). FIG. 19 (A and B) show HIV-1 VLP (consensus B and C) of S2 clones obtained from the upper and lower bands. As shown in these figures, virus particles obtained from the upper and lower bands have a complete sphere shape. Particle diameters are in a range of 96 nm-185 nm, and the average diameter of 125.7±23.2 nm (Table 4). Interestingly, HIV-1 VLP (consensus B and C) obtained from the upper band do not have surface spike (FIG. 19A) and HIV-1 VLP (consensus B and C) obtained from the lower band have envelope spikes observed on the surface (FIG. 19B). 12 HIV-1 VLP (consensus B and C) obtained from the lower band were selected for X-ray tomography test. It is found that each virus particle contained an average 17±2 spikes (ranging from 13-20) (FIG. 19C and Table 4). In addition, similar to that observed on the surface of SIV or HIV-1, the distance between surface spikes of HIV-1 VLP (consensus B and C) was also non-discrete (FIG. 19D).

TABLE 4
Purified HIV-1 VLP observed by tomographic imaging and
cryo-microscopy

| | | Envelope protein spike |
|---|---|---|
| HIV-1 VLP | Diameter (nm) | (number) |
| 1 | 128.380 | 18 |
| 2 | 142.229 | 19 |
| 3 | 134.887 | 18 |
| 4 | 123.321 | 17 |
| 5 | 101.999 | 14 |
| 6 | 114.596 | 13 |
| 7 | 128.440 | 20 |
| 8 | 124.155 | 17 |
| 9 | 96.270 | 20 |
| 10 | 111.778 | 14 |
| 11 | 186.633 | 20 |
| 12 | 115.479 | 18 |
| Mean ± standard | 125.681 ± 23.243 | 17 ± 2 |
| deviation | | |

Thus, the results show that gag protein and envelope protein expressed in the stably transfected S2 cells can be efficiently assembled into VLP, which is capable of being released from the cells. Its particle size is similar to that of wild type virus. Its gp160 precursor can be correctly cleaved into gp120 and gp41. It should be noted here that, although the present inventors have now used vectors with MT promoter and Ac5 promoter, many other promoters in Drosophila cells are, in fact, also suitable for expressing gag protein and envelope protein.

To determine the immunogenicity of HIV VLP, the present inventors compared two immunization strategies in mouse model using heterologous immunization (primary immunization with DNA and booster immunization with VLP) and homologous immunization (primary and booster immunization with DNA). In addition, they also compared the difference between homologous and heterologous immunization with and without adjuvant, such as CpG, ISA51, and ISA720.

The results show that:

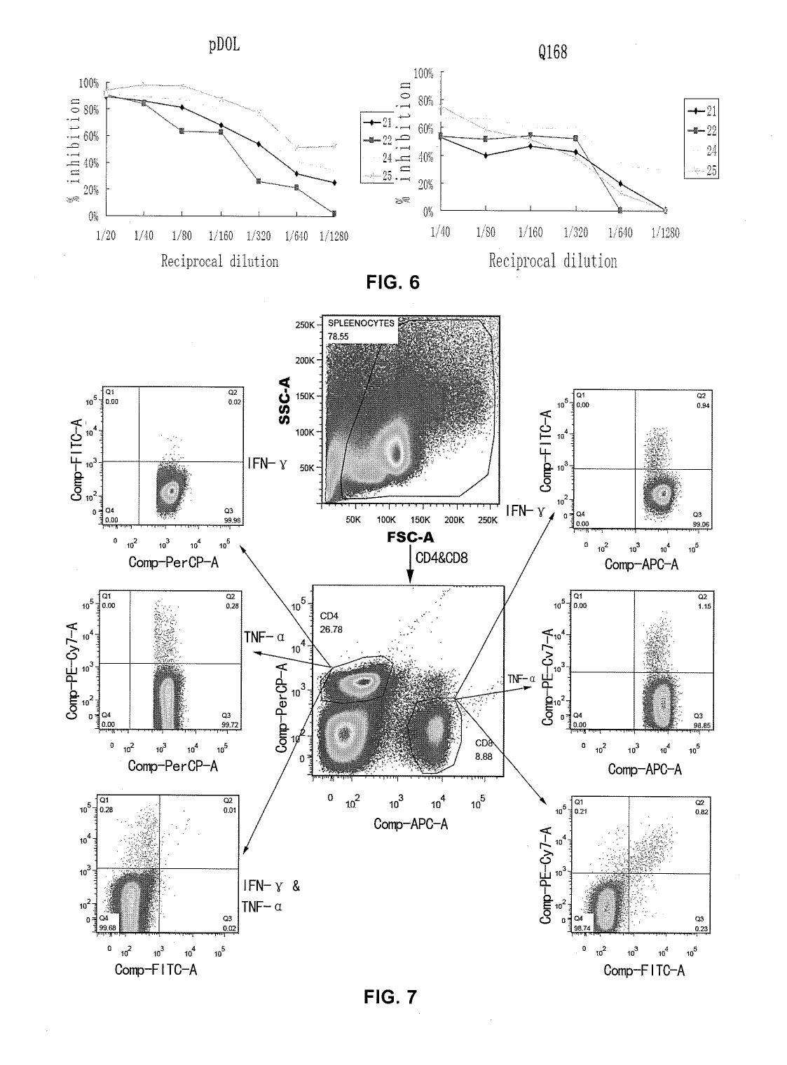

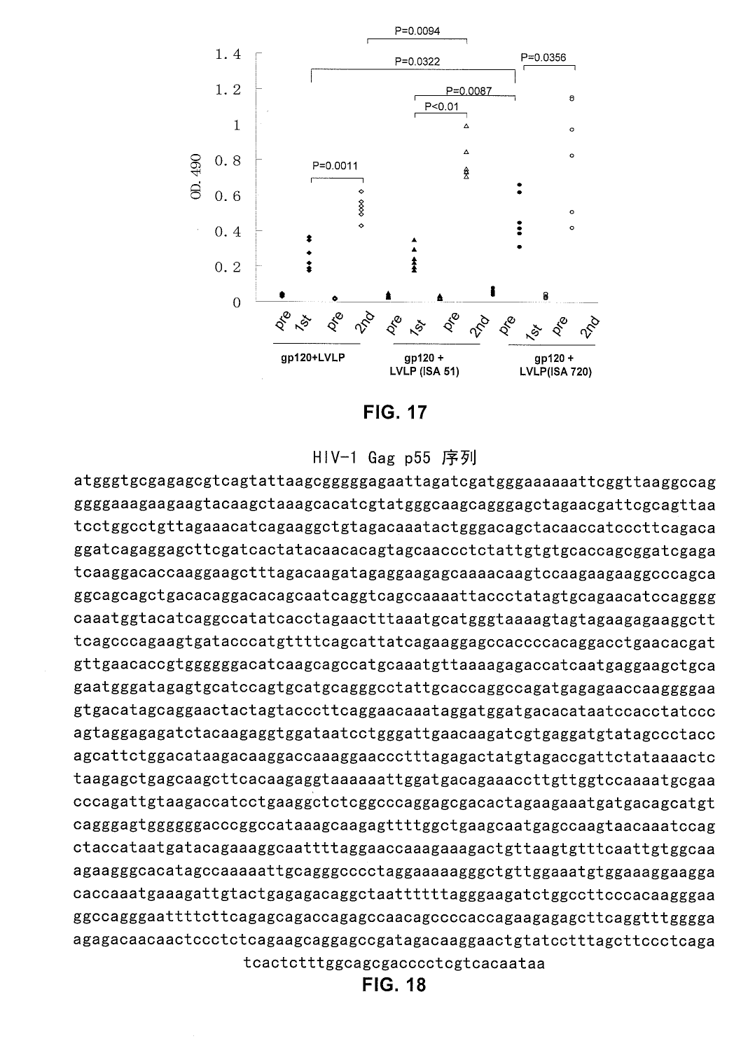

1) even in the absence of adjuvant, HIV VLP produced by S2 cells also has immunogenicity;
2) antibody response induced by VLP and adjuvant ISA720 is better than antibody response induced by VLP and adjuvant ISA51;
3) although all immunized groups can induce better ELISA antibody binding reaction (FIG. 17), but only the groups having primary immunization with DNA and booster immunization with VLP (obtained by expressing the co-transfected pMT-bip-HIVenv, pAC-HIVgag, pMT-HIVrev, and pCoBlast)/ISA720/CpG ODN show increased neutralizing antibody activity against homologous and against, limited, heterologous HIV-1 virus in mouse immune serum after the second booster immunization (FIG. 5 and FIG. 6);
4) immune response of CD8T and CD4T cells specific to HIV-1 envelope and gag peptides were detected in all mice having primary immunization with DNA and booster immunization with VLP. In addition, a majority of CD8T and CD4T cells specific to HIV-1 envelope and gag peptides simultaneously produced TNF alpha and IFN gamma (FIG. 7).
ADCC and ADCVI Response Induced by Heterologous Immunization with DNA-HIV-1 VLP (Consensus B and C)

To determine whether the immune serum produced by heterologous immunization with DNA-HIV-1 VLP (consensus B and C) can mediate ADCVI (antibody-dependent cell-mediated virus inhibition), first, HIV-1 AD8 was used to infect CEMss-CCR5 cells. 15 days later, viral replication was detected using HIV-1 gag p24 ELISA, followed by flow cytometry. And, pooled HIV-1 patient sera were used to detect cells expressing HIV-1 envelope protein on cell surface. We found that HIV-1 can replicate well in the infected CEMss-CCR5 cells. HIV-1 envelope protein can also be expressed on the surface of the infected cells (FIG. 20, right panel). Then, we used the sera from PBS immunized control mice and from DNA-HIV-1 VLP (consensus B and C) heterologously immunized mice to detect HIV-1 envelope protein expression on cell surface. The left panel and the middle panel of FIG. 20 show serum from mice heterologously immunized with DNA-HIV-1 VLP (consensus B and C) can recognize HIV-1 envelope protein on the surface of infected cells, but serum from the PBS immunized control mice cannot.

ADCC and ADCVI Response Induced by Heterologous Immunization with DNA-HIV-1 VLP (Consensus B and C)

To determine whether the immune serum produced by heterologous immunization with DNA-HIV-1 VLP (consensus B and C) can mediate ADCVI (antibody-dependent cell-mediated virus inhibition), first, HIV-1 AD8 was used to infect CEMss-CCR5 cells. 15 days later, viral replication was detected using HIV-1 gag p24 ELISA, followed by flow cytometry. And, pooled HIV-1 patient sera were used to detect cells expressing HIV-1 envelope protein on cell surface. We found that HIV-1 can replicate well in the infected CEMss-CCR5 cells. HIV-1 envelope protein can also be expressed on the surface of the infected cells (FIG. 20, right panel). Then, we used the sera from PBS immunized control mice and from DNA-HIV-1 VLP (consensus B and C) heterologously immunized mice to detect HIV-1 envelope protein expression on cell surface. The left panel and the middle panel of FIG. 20A show serum from mice heterologously immunized with DNA-HIV-1 VLP (consensus B and C) can recognize HIV-1 envelope protein on the surface of infected cells, but serum from the PBS immunized control mice cannot.

We then studied whether immune serum can mediate ADCC using AD8-infected cells as target cells and natural mouse cells as effector cells. FIG. 20B shows that 5-12% of ADCC activity, at 1:50 serum dilutions, was observed in immune serum from 6 immunized mice. However, there is no ADCC activity in serum of 6 PBS immunized control mice. The difference is statistically significant (P=0.0002). Similar results were obtained after repeating the experiment twice.

To further investigate ADCVI of immune serum, the identical AD8-infected cells were used as target cells and natural mouse cells as effector cells. FIG. 20C shows that 5-12% of ADCVI activity, at 1:50 serum dilutions, is observed in immune serum from 6 immunized mice. However, there is no ADCVI activity in serum of 6 PBS immunized control mice. The difference is statistically significant (P=0.006). Similar results were obtained after repeating the experiment twice. The death rate of ADCC and the inhibition rate of ADCVI were found to be positively correlated (r=0.9161) by analyzing all DNA-HIV-1 VLP (consensus B and C) heterologously immunized mice and PBS immunized control mice.

### Example 2

### Production of Influenza Virus VLP

To generate influenza virus VLP, the present inventors first compared the difference of the generated influenza virus VLP particles when influenza virus M1 protein and HIV-1 gag protein were used as core protein. Plasmid (pAC-M1) encoding influenza virus M1 protein inserted behind stable Ac5 promoter, and plasmids (pMT-bip-HA and pMT-bip-NA) encoding influenza virus HA and NA proteins inserted behind inducible MT promoter were constructed. As described above, S2 cells were co-transfected with these plasmids, i.e., pAC-M1, pMT-bip-HA, and pMT-bip-NA (used for forming HA-NA-M1 VLP); or pAC-HIVgag, pMT-bip-HA, and pMT-bip-NA (used for forming HA-NA-HIV-1 gag VLP), respectively, and a vector containing blasticidin resistance gene and the stably transfected S2 cells were selected. Then, the expression and assembly of the produced VLP were investigated.

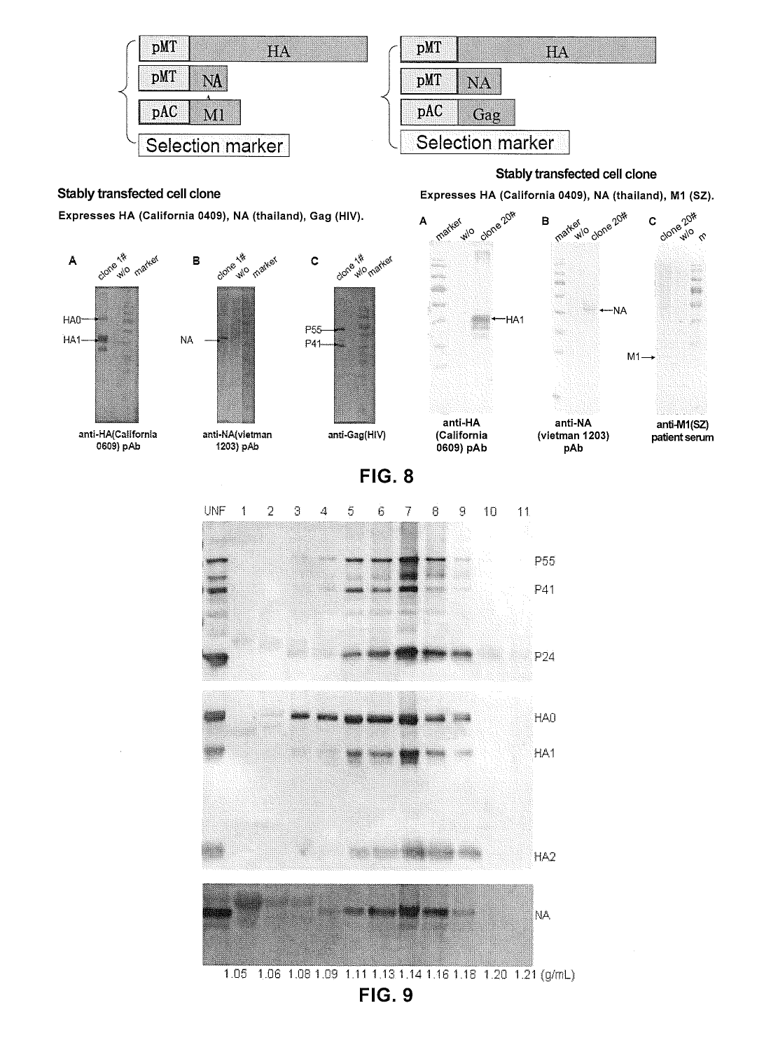

FIG. 8 shows the expression of HA, NA, M1, and HIV-1 gag protein in cell lysates and cell culture supernatants of stably transfected cells with or without CdCl₂ induction. The results show all HA, NA, M1 or HA, NA, and HIV-1 gag are normally expressed, and HA can be correctly cleaved.

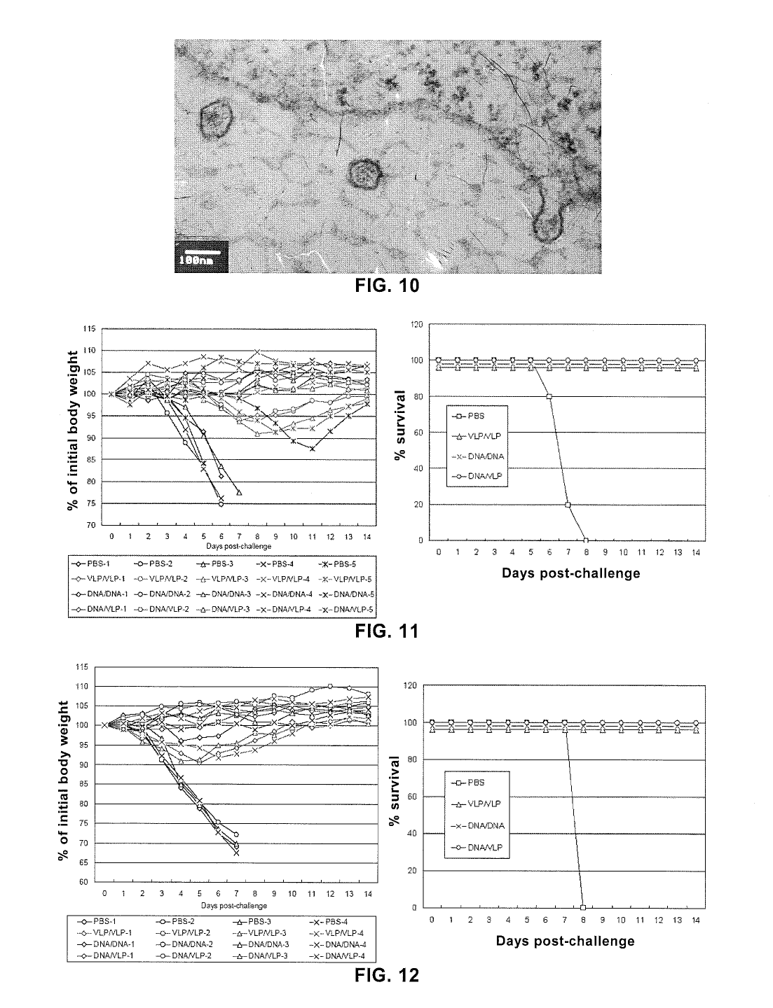

In the subsequent studies, the present inventors put research focus on producing influenza virus VLP (HA-NA-HIV-1 gag VLP) having HIV-1 gag as core protein. FIG. 9 shows the characteristics of influenza virus VLP after sucrose density gradient centrifugation and Western blot analysis. FIG. 10 shows electron microscopic image of influenza virus VLP. The diameter of VLP particles is within a range of 80-120 nm.

Thus, the results show that gag protein, HA, and NA envelope protein expressed in the stably transfected S2 cells can be effectively assembled into VLP, and capable of being released from cells. Its particle size is very similar to that of wild type virus, and its HA₀ precursor can be correctly cleaved into HA₁ and HA₂.

To determine the immunogenicity of influenza virus VLP, the present inventors compared the neutralizing antibody response and immune protection induced by homologous immunization strategy of DNA-DNA and VLP-VLP, and heterologous immunization strategy of DNA-VLP. The results show that heterologous immunization strategy of DNA-VLP can induce the best neutralizing antibody titers against homologous and heterologous influenza virus H5N1 (Table 2 and Table 3).

TABLE 2
Neutralizing titers of serum sample for homologous viruses

| | | Pre-prime sera | | Post-boost sera | | Post-challenge sera | |
|---|---|---|---|---|---|---|---|
| | NO | IC50 | IC95 | IC50 | IC95 | IC50 | IC95 |
| PBS | 1 | >1:10* | >1:10 | >1:10 | >1:10 | ND** | ND |
| | 2 | >1:10 | >1:10 | >1:10 | >1:10 | ND | ND |
| | 3 | >1:10 | >1:10 | >1:10 | >1:10 | ND | ND |
| | 4 | >1:10 | >1:10 | >1:10 | >1:10 | ND | ND |
| | 5 | >1:10 | >1:10 | >1:10 | >1:10 | ND | ND |
| | 6 | >1:10 | >1:10 | >1:10 | >1:10 | ND | ND |
| VLP/VLP | 1 | >1:10 | >1:10 | 1:640-1:2560 | >1:10 | 1:640-1:2560 | 1:40-1:160 |
| | 2 | >1:10 | >1:10 | 1:40-1:160 | >1:10 | 1:640-1:2560 | 1:10-1:40 |
| | 3 | >1:10 | >1:10 | 1:10-1:40 | >1:10 | 1:640-1:2560 | 1:10-1:40 |
| | 4 | >1:10 | >1:10 | 1:40-1:160 | >1:10 | 1:640-1:2560 | 1:40-1:160 |
| | 5 | >1:10 | >1:10 | 1:640-1:2560 | >1:10 | 1:640-1:2560 | 1:40-1:160 |
| | 6 | >1:10 | >1:10 | 1:160-1:640 | >1:10 | ND | ND |
| DNA/DNA | 1 | >1:10 | >1:10 | 1:640-1:2560 | 1:10-1:40 | 1:2560 | 1:160-1:640 |
| | 2 | >1:10 | >1:10 | 1:640-1:2560 | 1:40-1:160 | 1:640-1:2560 | 1:40-1:160 |
| | 3 | >1:10 | >1:10 | 1:640-1:2560 | 1:10-1:40 | 1:640-1:2560 | 1:40-1:160 |
| | 4 | >1:10 | >1:10 | 1:640-1:2560 | 1:10-1:40 | 1:640-1:2560 | 1:40-1:160 |
| | 5 | >1:10 | >1:10 | 1:160-1:640 | >1:10 | 1:640-1:2560 | 1:40-1:160 |
| | 6 | >1:10 | >1:10 | 1:2560-1:10240 | 1:10-1:40 | ND | ND |
| DNA/VLP | 1 | >1:10 | >1:10 | 1:2560-1:10240 | 1:40-1:160 | 1:10240 | 1:160-1:640 |
| | 2 | >1:10 | >1:10 | 1:2560-1:10240 | 1:40-1:160 | 1:2560-1:10240 | 1:160-1:640 |
| | 3 | >1:10 | >1:10 | 1:2560-1:10240 | 1:40-1:160 | 1:2560-1:10240 | 1:40-1:160 |
| | 4 | >1:10 | >1:10 | 1:2560-1:10240 | 1:40-1:160 | 1:2560-1:10240 | 1:160-1:640 |
| | 5 | >1:10 | >1:10 | 1:2560-1:10240 | 1:40-1:160 | 1:10240 | 1:160-1:640 |
| | 6 | >1:10 | >1:10 | 1:2560-1:10240 | 1:40-1:160 | ND | ND |

In which, Pre-prime sera indicates sera obtained from the first blood drawn; Post-boost sera indicates sera obtained from blood drawn after booster immunization; Post-challenge sera indicates sera obtained from after infection.

TABLE 3
Neutralizing titers of serum sample for heterologous viruses

| | | Pre-prime | | Post-boost | | | | |
|---|---|---|---|---|---|---|---|---|
| | | sera | | sera | | Post-challenge sera | | |
| | NO | IC50 | IC95 | IC50 | IC95 | IC50 | IC90 | IC95 |
| PBS | 1 | >1:10* | >1:10 | >1:10 | >1:10 | ND** | ND | ND |
| | 2 | >1:10 | >1:10 | >1:10 | >1:10 | >1:10 | >1:10 | >1:10 |
| | 3 | >1:10 | >1:10 | >1:10 | >1:10 | ND | ND | ND |
| | 4 | >1:10 | >1:10 | >1:10 | >1:10 | ND | ND | ND |
| VLP/VLP | 1 | >1:10 | >1:10 | >1:10 | >1:10 | 1:40-1:160 | >1:10 | >1:10 |
| | 2 | >1:10 | >1:10 | >1:10 | >1:10 | 1:160-1:640 | 1:10-1:40 | >1:10 |
| | 3 | >1:10 | >1:10 | >1:10 | >1:10 | 1:40-1:160 | >1:10 | >1:10 |
| | 4 | >1:10 | >1:10 | >1:10 | >1:10 | 1:40-1:160 | >1:10 | >1:10 |
| DNA/DNA | 1 | >1:10 | >1:10 | >1:10 | >1:10 | 1:160-1:640 | 1:10-1:40 | >1:10 |
| | 2 | >1:10 | >1:10 | >1:10 | >1:10 | 1:160-1:640 | 1:10 | >1:10 |
| | 3 | >1:10 | >1:10 | >1:10 | >1:10 | 1:160-1:640 | 1:10-1:40 | >1:10 |
| | 4 | >1:10 | >1:10 | >1:10 | >1:10 | 1:160 | >1:10 | >1:10 |
| DNA/VLP | 1 | >1:10 | >1:10 | >1:10 | >1:10 | 1:640-1:2560 | 1:40-1:160 | 1:10-1:40 |
| | 2 | >1:10 | >1:10 | >1:10 | >1:10 | 1:640 | 1:10-1:40 | >1:10 |
| | 3 | >1:10 | >1:10 | >1:10 | >1:10 | 1:640 | 1:10-1:40 | >1:10 |
| | 4 | >1:10 | >1:10 | >1:10 | >1:10 | 1:160-1:640 | 1:10-1:40 | >1:10 |

In addition, the findings of the present inventors show that, although all three immunization strategies can protect mice to resist challenges by 10 MLD50 of homologous or heterologous H5N1 virus, but only DNA-DNA and DNA-VLP immunization strategy can protect mice to resist challenges by 1000 MLD50 of homologous H5N1 (FIGS. 11-13).

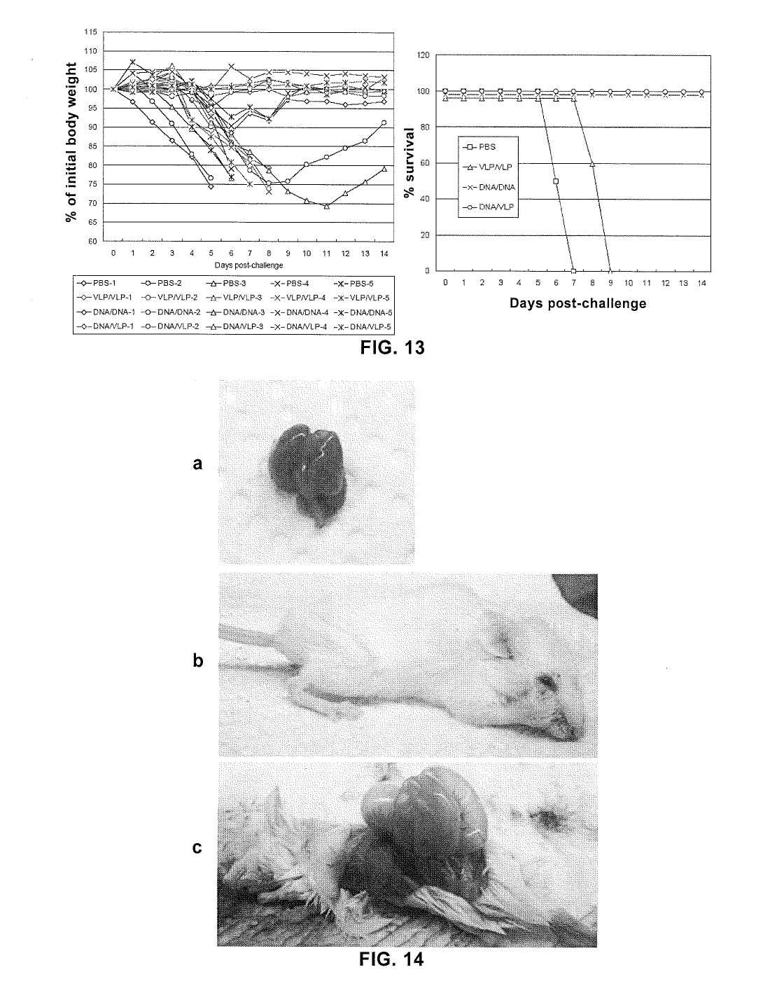

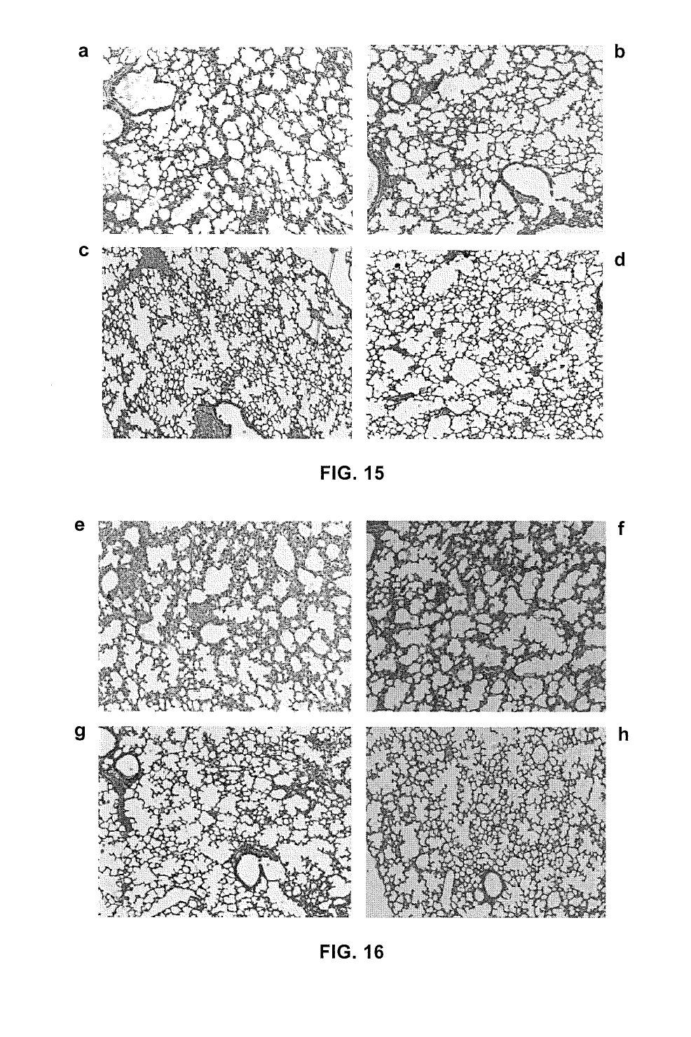

The findings of the present inventors also show that only DNA-VLP immunization strategy can completely protect mice from sickness after challenges by 10 MLD50 and 1000 MLD50 of H5N1 (FIGS. 11-16). Thus, the findings of the present inventors confirm that heterologous DNA-VLP immunization strategy can be used for human immunization to prevent possible pandemic of H5N1 outbreak.

In addition to producing the above influenza virus VLP expressing sub-branch 2.3.4 H5HA and NINA, the present inventors also prepared four kinds of influenza virus VLP expressing new pandemic strain H1N1, sub-branch 2.3.4 H5HA, two kinds of H5HA in branch 1, etc.

All literature mentioned in the present invention are incorporated by reference in the present application, as if each reference were individually incorporated by reference. It should also be understood, after reading the disclosure of the present invention, one skilled in the art could make various changes and modifications to the present invention. These equivalence similarly fall within the scope limited by the appended claims in the present application.

1. A method of producing a virus-like particle of an enveloped virus, characterized in that the method comprises:
transforming a Drosophila cell with a nucleic acid comprising a sequence encoding an antigenic protein of the enveloped virus to obtain a recombinant virus-like particle producing cell;
culturing the recombinant virus-like particle producing cell to express and obtain the virus-like particle,
wherein the Drosophila cell is a Drosophila melanogaster S2 cell.

2. The method of claim 1, characterized in that the nucleic acid comprises a nucleic acid encoding a viral core protein and a nucleic acid encoding an antigenic protein of the enveloped virus.

3. The method of claim 2, characterized in that the method comprises:
(A) providing a first expression construct, comprising a nucleic acid sequence encoding the viral core protein;
(B) providing a second expression construct, comprising a nucleic acid sequence encoding the antigenic protein of the enveloped virus;
(C) transforming the Drosophila melanogaster S2 cell with the constructs of (A) and (B), to obtain the recombinant virus-like particle producing cell; and
(D) culturing the recombinant virus-like particle producing cell of (C) recombinant virus-like particle production cells to express and obtain the virus-like particle.

4. The method of claim 2, characterized in that the viral core protein is selected from: Gag protein of human immunodeficiency virus, M1 protein of influenza virus, Gag protein of simian immunodeficiency virus, Gag viral core protein of murine leukemia virus, M viral core protein of vesicular stomatitis virus, VP40 viral core protein of Ebola virus, M and E proteins of coronavirus, N protein of Bunia virus, core protein C of hepatitis C virus, core protein of hepatitis B virus, core protein of SARS coronavirus, and a combination thereof.

5. (canceled)

6. The method of claim 1, characterized in that the virus comprises influenza virus, human immunodeficiency virus, paramyxovirus, Borna disease virus, rabies virus, and Ebola virus.

7. (canceled)

8. The method of claim 3, characterized in that the expression vector is a non-viral vector.

9. The method of claim 8, characterized in that the expression vector comprises a Drosophila promoter selected from: MT promoter or Ac5 promoter.

10. The method of claim 8, characterized in that the non-viral vector is selected from: pMT/V5-His, pMT/BiP/V5-His, pMT-DEST48 or pMT/V5-His-TOPO.

11. The method of claim 1, characterized in that further comprises: transforming the Drosophila S2 cell with a nucleic acid encoding a regulator of expression of virion protein.

12. The method of claim 1, characterized in that further comprises: transforming the Drosophila S2 cell with a resistance selection gene.

13. The method of claim 1, characterized in that the virus-like particle is a virus-like particles derived from influenza virus, the method comprising:
(A1) providing a first expression construct, comprising a nucleic acid sequence encoding Gag protein of human immunodeficiency virus or a nucleic acid sequence encoding M1 protein of influenza virus;
(B1) providing a second expression construct, comprising a nucleic acid sequence encoding neuraminidase antigen of influenza virus and/or a nucleic acid sequence encoding hemagglutinin antigen of influenza virus;
(C1) transforming the Drosophila S2 cell with the constructs of (A1) and (B1), obtaining a recombinant virus-like particle producing cell; and
(D1) culturing the recombinant virus-like particle producing cell of (C1), from which expressing and obtaining the virus-like particle;
wherein the first expression construct and the second expression construct are located on an expression vector; or the first expression construct and the second expression construct are located on different expression vectors.

14. The method of claim 13, characterized in that the nucleic acid sequence encoding neuraminidase antigen of influenza virus and the nucleic acid sequence encoding hemagglutinin antigen of influenza virus of the second expression construct of the step (B1) are located on the same expression vector or located on different expression vectors.

15. The method of claim 1, characterized in that the virus-like particle is a virus-like particle derived from human immunodeficiency virus, the method comprising:
(A2) providing a first expression construct, comprising a nucleic acid sequence encoding Gag protein of human immunodeficiency virus;
(B2) providing a second expression construct, comprising a nucleic acid sequence encoding envelope protein precursor Gp160 of human immunodeficiency virus;
(C2) transforming Drosophila S2 cells with the constructs of (A2) and (B2), obtaining a recombinant virus-like particle producing cell; and
(D2) culturing the recombinant virus-like particle producing cell of (C2), from which expressing and obtaining the virus-like particle;
wherein the first expression construct and the second expression construct are located on an expression vector; or the first expression construct and the second expression construct are located on different expression vectors.

16. (canceled)

17. A method, using the virus-like particle produced by the method of claim 1, for preventing, controlling or treating one of the following diseases, disorders, or conditions: influenza, HIV, measles, respiratory syncytial virus infection, mumps, pneumonia virus infection, Borna disease, rabies, and Ebola haemorrhagic fever.

18. (canceled)

19. (canceled)

20. (canceled)

21. (canceled)

22. (canceled)

23. A Drosophila cell, characterized in that the Drosophila cell comprising a vector for producing a virus-like particle of an enveloped virus, wherein the Drosophila cell is a Drosophila melanogaster S2 cell.

24. The Drosophila cell of claim 23, characterized in that the vector for producing the virus-like particle of the enveloped virus comprising a nucleic acid encoding a viral core protein and a nucleic acid encoding an antigenic protein of an enveloped virus.


SEQUENCE LISTING (15 sequences)

SEQ ID NO: 1 - DNA, 1503 bp, Human immunodeficiency virus
atgggtgcgagagcgtcagtattaagcgggggagaattagatcgatgggaaaaaattcggttaaggccagggggaaagaagaagtacaagctaaagcacatcgtatgggcaagcagggagctagaacgattcgcagttaatcctggcctgttagaaacatcagaaggctgtagacaaatactgggacagctacaaccatcccttcagacaggatcagaggagcttcgatcactatacaacacagtagcaaccctctattgtgtgcaccagcggatcgagatcaaggacaccaaggaagctttagacaagatagaggaagagcaaaacaagtccaagaagaaggcccagcaggcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaggacctgaacacgatgttgaacaccgtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagagatctacaagaggtggataatcctgggattgaacaagatcgtgaggatgtatagccctaccagcattctggacataagacaaggaccaaaggaaccctttagagactatgtagaccgattctataaaactctaagagctgagcaagcttcacaagaggtaaaaaattggatgacagaaaccttgttggtccaaaatgcgaacccagattgtaagaccatcctgaaggctctcggcccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataa

SEQ ID NO: 2 - DNA, 26 bp, Artifical sequence
  misc_feature: Primer
ctagaattcaacagagaagctgtggg

SEQ ID NO: 3 - DNA, 20 bp, Artifical sequence
  misc_feature: Primer
gatgcggccgcttactttcc

SEQ ID NO: 4 - DNA, 26 bp, Artifical sequence
  misc_feature: Primer
ctagaattcaccatggcaggaagaag

SEQ ID NO: 5 - DNA, 23 bp, Artifical sequence
  misc_feature: Primer
agtgcggccgcctattctttagc

SEQ ID NO: 6 - DNA, 22 bp, Artifical sequence
  misc_feature: Primer
gtcgaattcaccatgggtgcga

SEQ ID NO: 7 - DNA, 22 bp, Artifical sequence
  misc_feature: Primer
gtagcggccgcttattgtgacg

SEQ ID NO: 8 - DNA, 25 bp, Artifical sequence
  misc_feature: Primer
gggagatcttgcatcggataccacg

SEQ ID NO: 9 - DNA, 26 bp, Artifical sequence
  misc_feature: Primer
cccgaattctcagatgcagattctgc

SEQ ID NO: 10 - DNA, 32 bp, Artifical sequence
  misc_feature: Primer
gggggatccatgaatcctaataagaagatcat

SEQ ID NO: 11 - DNA, 33 bp, Artifical sequence
  misc_feature: Primer
cccgaattcctcacttatcgattgtaaaaggca

SEQ ID NO: 12 - DNA, 31 bp, Artifical sequence
  misc_feature: Primer
cgggaattcaccatgagtcttctaaccgagg

SEQ ID NO: 13 - DNA, 30 bp, Artifical sequence
  misc_feature: Primer
ccctctagatcacttgaatcgctgcatctg

SEQ ID NO: 14 - DNA, 2553 bp, Human immunodeficiency virus
atgcgcgtgaagggcatccgcaagaactaccagcacctgtggcgctggggcaccatgctgctgggcatgctgatgatctgctccgccgccgagaagctgtgggtgaccgtgtactacggcgtgcccgtgtggaaggaggccaccaccaccctgttctgcgcctccgacgccaaggcctacgacaccgaggtgcacaacgtgtgggccacccacgcctgcgtgcccaccgaccccaacccccaggaggtggtgctggagaacgtgaccgagaacttcaacatgtggaagaacaacatggtggagcagatgcacgaggacatcatctccctgtgggaccagtccctgaagccctgcgtgaagctgacccccctgtgcgtgaccctgaactgcaccgacctgaagaacaacctgctgaacaccaactcctcctccggcgagaagatggagaagggcgagatcaagaactgctccttcaacatcaccacctccatccgcgacaaggtgcagaaggagtacgccctgttctacaagctggacgtggtgcccatcgacaacaacaacaacacctcctaccgcctgatctcctgcaacacctccgtgatcacccaggcctgccccaaggtgtccttcgagcccatccccatccactactgcgcccccgccggcttcgccatcctgaagtgcaacgacaagaagttcaacggcaccggcccctgcaccaacgtgtccaccgtgcagtgcacccacggcatccgccccgtggtgtccacccagctgctgctgaacggctccctggccgaggaggaggtggtgatccgctccgagaacttcaccgacaacgccaagaccatcatcgtgcagctgaacgagtccgtggagatcaactgcacccgccccaacaacaacacccgcaagtccatccacatcggccccggccgcgccttctacaccaccggcgagatcatcggcgacatccgccaggcccactgcaacatctcccgcgccaagtggaacaacaccctgaagcagatcgtgaagaagctgcgcgagcagttcggcaacaagaccatcgtgttcaaccagtcctccggcggcgaccccgagatcgtgatgcactccttcaactgcggcggcgagttcttctactgcaacaccacccagctgttcaactccacctggaacgacaacggcacctggaacaacaccaaggacaagaacaccatcaccctgccctgccgcatcaagcagatcatcaacatgtggcaggaggtgggcaaggccatgtacgccccccccatccgcggccagatccgctgctcctccaacatcaccggcctgctgctgacccgcgacggcggcaacaacaacaacgacaccgagatcttccgccccggcggcggcgacatgcgcgacaactggcgctccgagctgtacaagtacaaggtggtgaagatcgagcccctgggcgtggcccccaccaaggccaagcgccgcgtggtgcagcgcgagaagcgcgccgtgggcatcggcgccatgttcctgggcttcctgggcgccgccggctccaccatgggcgccgcctccatgaccctgaccgtgcaggcccgccagctgctgtccggcatcgtgcagcagcagaacaacctgctgcgcgccatcgaggcccagcagcacctgctgcagctgaccgtgtggggcatcaagcagctgcaggcccgcgtgctggccgtggagcgctacctgaaggaccagcagctgctgggcatctggggctgctccggcaagctgatctgcaccaccaccgtgccctggaacgcctcctggtccaacaagtccctggacgagatctgggacaacatgacctggatggagtgggagcgcgagatcgacaactacacctccctgatctacaccctgatcgaggagtcccagaaccagcaggagaagaacgagcaggagctgctggagctggacaagtgggcctccctgtggaactggttcgacatcaccaactggctgtggtacatcaagatcttcatcatgatcgtgggcggcctgatcggcctgcgcatcgtgttcgccgtgctgtccatcgtgaaccgcgtgcgccagggctactcccccctgtccttccagacccgcctgcccgccccccgcggccccgaccgccccgagggcatcgaggaggagggcggcgagcgcgaccgcgaccgctccggccgcctggtggacggcttcctggccctgatctgggacgacctgcgctccctgtgcctgttctcctaccaccgcctgcgcgacctgctgctgatcgtgacccgcatcgtggagctgctgggccgccgcggctgggaggtgctgaagtactggtggaacctgctgcagtactggtcccaggagctgaagaactccgccgtgtccctgctgaacgccaccgccatcgccgtggccgagggcaccgaccgcgtgatcgaggtggtgcagcgcgcctgccgcgccatcctgcacatcccccgccgcatccgccagggcctggagcgcgccctgctgtaa

SEQ ID NO: 15 - DNA, 2529 bp, Human immunodeficiency virus
atgcgcgtgatgggcatcctgcgcaactgccagcagtggtggatctggggcatcctgggcttctggatgctgatgatctgcaacgtggtgggcaacctgtgggtgaccgtgtactacggcgtgcccgtgtggaaggaggccaagaccaccctgttctgcgcctccgacgccaaggcctacgagaaggaggtgcacaacgtgtgggccacccacgcctgcgtgcccaccgaccccaacccccaggagatggtgctggagaacgtgaccgagaacttcaacatgtggaagaacgacatggtggaccagatgcacgaggacatcatctccctgtgggaccagtccctgaagccctgcgtgaagctgacccccctgtgcgtgaccctgaactgccgcaacgtgaccaacgccaccaacaacacctacaacgaggagatcaagaactgctccttcaacatcaccaccgagctgcgcgacaagaagaagaaggtgtacgccctgttctaccgcctggacatcgtgcccctgaacgagaactcctccgagtaccgcctgatcaactgcaacacctccgccatcacccaggcctgccccaaggtgtccttcgaccccatccccatccactactgcgcccccgccggctacgccatcctgaagtgcaacaacaagaccttcaacggcaccggcccctgcaacaacgtgtccaccgtgcagtgcacccacggcatcaagcccgtggtgtccacccagctgctgctgaacggctccctggccgaggaggagatcatcatccgctccgagaacctgaccaacaacgccaagaccatcatcgtgcacctgaacgagtccgtggagatcgtgtgcacccgccccaacaacaacacccgcaagtccatccgcatcggccccggccagaccttctacgccaccggcgacatcatcggcgacatccgccaggcccactgcaacatctccgaggacaagtggaacaagaccctgcagcgcgtgtccaagaagctgaaggagcacttccccaacaagaccatcaagttcgagccctcctccggcggcgacctggagatcaccacccactccttcaactgccgcggcgagttcttctactgcaacacctccaagctgttcaactccacctacaacaacaacaccaactccaactccaccatcaccctgccctgccgcatcaagcagatcatcaacatgtggcaggaggtgggccgcgccatgtacgccccccccatcgccggcaacatcacctgcaagtccaacatcaccggcctgctgctgacccgcgacggcggcaagaagaacaccaccgagatcttccgccccggcggcggcgacatgcgcgacaactggcgctccgagctgtacaagtacaaggtggtggagatcaagcccctgggcgtggcccccaccaaggccaagcgccgcgtggtggagcgcgagaagcgcgccgtgggcatcggcgccgtgttcctgggcttcctgggcgccgccggctccaccatgggcgccgcctccatcaccctgaccgtgcaggcccgccagctgctgtccggcatcgtgcagcagcagtccaacctgctgcgcgccatcgaggcccagcagcacatgctgcagctgaccgtgtggggcatcaagcagctgcagacccgcgtgctggccatcgagcgctacctgaaggaccagcagctgctgggcatctggggctgctccggcaagctgatctgcaccaccgccgtgccctggaactcctcctggtccaacaagtcccaggaggacatctgggacaacatgacctggatgcagtgggaccgcgagatctccaactacaccgacaccatctaccgcctgctggaggactcccagaaccagcaggagaagaacgagaaggacctgctggccctggactcctggaagaacctgtggaactggttcgacatcaccaactggctgtggtacatcaagatcttcatcatgatcgtgggcggcctgatcggcctgcgcatcatcttcgccgtgctgtccatcgtgaaccgcgtgcgccagggctactcccccctgtccttccagaccctgacccccaacccccgcggccccgaccgcctgggccgcatcgaggaggagggcggcgagcaggaccgcgaccgctccatccgcctggtgtccggcttcctggccctggcctgggacgacctgcgctccctgtgcctgttctcctaccaccgcctgcgcgacttcatcctggtggccgcccgcgccgtggagctgctgggccgctcctccctgcgcggcctgcagcgcggctgggaggccctgaagtacctgggctccctggtgcagtactggggcctggagctgaagaagtccgccatctccctgctggacaccatcgccatcgccgtggccgagggcaccgaccgcatcatcgagctgatccagcgcatctgccgcgccatccgcaacatcccccgccgcatccgccagggcttcgaggccgccctgcagtaa